(19) United States
(12) Patent Application Publication
(10) Pub. No.: US 2025/0264468 A1
(43) Pub. Date: Aug. 21, 2025

(54) COMPOSITIONS AND METHODS FOR THE DETECTION OF COCCIDIOIDAL CHITINASE-1

(71) Applicants: Mayo Foundation for Medical Education and Research, Rochester, MN (US); Arizona Board of Regents Acting for and on Behalf of Arizona State University, Scottsdale, AZ (US)

(72) Inventors: Douglas Lake, Scottsdale, AZ (US); Thomas E. Grys, Scottsdale, AZ (US); Francisca J. Grill, Scottsdale, AZ (US)

(21) Appl. No.: 18/697,663

(22) PCT Filed: Sep. 29, 2022
(86) PCT No.: PCT/US22/77281

§ 371 Date: Apr. 1, 2024

Related U.S. Application Data
(60) Provisional application No. 63/331,077, filed on Apr. 14, 2022.
(60) Provisional application No. 63/251,243, filed on Oct. 1, 2021.

(51) Int. Cl. G01N 33/573; G01N 33/569

(52) U.S. Cl. CPC G01N 33/573; G01N 33/56961; G01N 2333/37; G01N 2333/942

(57) ABSTRACT

The present disclosure provides compositions and methods related to the detection of coccidioidomycosis (Valley fever). In particular, the present disclosure provides novel compositions and methods related to the detection and/or quantification of coccidioidomycosis in a subject using an enzyme-linked immunoassay (ELISA) that measures coccidioidal CTS1 antigen and/or anti-CTS1 antibodies in human serum.

### CROSS REFERENCE TO RELATED APPLICATIONS

This application is a National Stage application under 35 U.S.C. § 371 of International Application No. PCT/US2022/077281 having an international filing date of Sep. 29, 2022, which claims priority to and the benefit of U.S. Provisional Patent Application No. 63/251,243 filed Oct. 1, 2021, and U.S. Provisional Patent Application No. 63/331,077 filed Apr. 14, 2022, both of which are incorporated herein by reference in their entireties and for all purposes.

### GOVERNMENT FUNDING

This invention was made with government support under A1152042 awarded by the National Institutes of Health. The government has certain rights in the invention.

### INCORPORATION-BY-REFERENCE OF MATERIAL SUBMITTED ELECTRONICALLY

This application contains a Sequence Listing that has been submitted electronically as an XML file named “SKYSG-39948-601.” The XML file, created on Sep. 29, 2022, is 13,108 bytes in size. The material in the XML file is hereby incorporated by reference in its entirety.

### FIELD

The present disclosure provides compositions and methods related to the detection of coccidioidomycosis (Valley fever). In particular, the present disclosure provides novel compositions and methods related to the detection and/or quantification of coccidioidomycosis in a subject using an enzyme-linked immunoassay (ELISA) that measures coccidioidal CTS1 antigen and/or anti-CTS1 antibodies in human serum.

### BACKGROUND

Coccidioidomycosis, or Valley Fever (VF), is an infection caused by the pathogenic fungi Coccidioides immitis and Coccidioides posadasii. Coccidioides spp. inhabit arid areas throughout the Americas including Arizona and California, both of which have experienced a significant increase in incidence since 2014. Infection may be asymptomatic or manifest as a pneumonia difficult to distinguish from community-acquired viral or bacterial pneumonia, which may lead to inappropriate treatment. In a small but significant number of cases, extra-pulmonary dissemination of the fungus occurs resulting in significant morbidity, need for long-term antifungal therapy, and sometimes in death.

Coccidioides spp. are dimorphic and can exist in two forms dependent on their environment. In the soil, Coccidioides grow as septate mycelia composed of arthroconidia that become aerosolized upon disruption and are easily inhaled into the lungs of a human or animal host. In the host, the fungus transforms into its spherule growth form that undergoes maturation, internally dividing and producing endospores. Production of chitin, a major component in the cell wall of spherules, increases significantly during the beginning of the endosporulation process. This surge is then diminished with the progression of endospore formation and release, at which time chitinases are detectable. Chitinase 1 (CTS1), is a 48-kDa protein that plays a role in weakening the spherule cell wall prior to endospore release. In culture, the presence of CTS1 is shown to decrease shortly after endosporulation due to an upregulation of proteases, so the process of spherule growth and endosporulation can restart.

CTS1 is also known as “CF” antigen used in serodiagnostics, namely (CF) assays and its immunodiffusion correspondent (IDCF). CTS1 is also utilized in enzyme immunoassay (EIA) formats. EIA and ID can distinguish between IgM and IgG antibodies, while CF can quantify the antibody response and measure disease progression and/or regression. The sensitivity and specificity of these assays in diagnosis have been evaluated by multiple groups, and although helpful, the sensitivity of serology is still lacking, especially in early infection and in immunosuppressed individuals. Specificity can also be a problem. Several groups have recombinantly produced and characterized CTS1 in an effort to increase sensitivity and specificity for detecting antibodies in patients. Furthermore, it can take weeks to months after onset of symptoms to detect an immune response. Unlike many serologic assays for infectious diseases, IgG is not a marker of distant infection but instead used to diagnose recent or latent infection. As disease resolves, the antibodies detected by clinical assays wane. Alternatively, resolution of fungal infection may be incomplete (latent) or unconfirmed due to a long period of detectable antibody. Thus, while serology continues to be a mainstay diagnostic tool, it is an incomplete and imperfect approach.

Morphological and growth-based diagnostics such as microscopy and culture, respectively, exist, but both are lacking in sensitivity and the latter poses a risk to laboratory personnel despite being considered the gold-standard of diagnosis. Polymerase chain reaction (PCR) for Coccidioides has been developed but its sensitivity is similar to that of culture. Detection of coccidioidal antigens has been approached previously using spherule lysate, and more recently using galactomannan. No follow up evaluations of the former have been explored, but the latter has shown value in diagnosing more severe forms of disease such as coccidioidal meningitis. A recent publication affirmed the modest incremental value of performing the Coccidioides galactomannan antigen assay, though the assay was positive for only a minority of non-disseminated cases. This experience has therefore highlighted the need for a sensitive antigen-based diagnostic test that detects and/or measures direct biomarkers from Coccidioides spp. as opposed to a patient response. Embodiments of the present disclosure describe the development of a new enzyme-linked immunoassay (ELISA) that measures coccidioidal CTS1 antigen in commercial antigen preparations. While its ability to exclusively quantify antigen in human serum is equivocal due to interference from endogenous antibodies, the clinical sensitivity to detect active infection appears superior to existing serologic methods.

### SUMMARY

Embodiments of the present disclosure include a composition for detecting and/or quantifying Coccidioidal Chitinase-1 (CTS1) antigen and/or anti-CTS1 antibodies. In accordance with these embodiments, the composition includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the composition further comprises a sample from a subject, wherein: (a) CTS1 present in the sample inhibits binding of the CTS1 detection agent to the recombinant CTS1 polypeptide, thereby facilitating detection of the CTS1 in the sample; and/or (b) anti-CTS1 antibodies present in the sample inhibit binding of the CTS1 detection agent to the recombinant CTS1 polypeptide, thereby facilitating detection of the anti-CTS1 antibodies in the sample.

In some embodiments, the CTS1 detection agent comprises a CTS1 antibody or a CTS1 target antigen binding fragment thereof.

In some embodiments, the detection moiety comprises at least one of a peptide, a polypeptide, a fluorophore, a chromophore, a radiolabel, a nucleotide, a polynucleotide, a small molecule, an enzyme, and/or a nanoparticle. In some embodiments, the small molecule is biotin. In some embodiments, the polypeptide is streptavidin.

In some embodiments, the sample is a serum sample from a subject that is infected with or suspected of being infected with Coccidioides spp.

In some embodiments, the recombinant CTS1 polypeptide is attached to a substrate. In some embodiments, the substrate comprises glass, silicon, a metal oxide, and/or a polymer.

In some embodiments, the composition further comprises a CTS1 calibrator or control polypeptide.

Embodiments of the present disclosure also include a method for assessing a sample from a subject for a coccidioidomycosis infection. In accordance with these embodiments, the method includes performing a reaction to detect the CTS1 detection agent bound to the recombinant CTS1 polypeptide in any of the compositions described herein, wherein CTS1 present in the sample inhibits binding of the CTS1 detection agent to the recombinant CTS1 polypeptide, thereby indicating the presence of CTS1 in the sample.

Embodiments of the present disclosure also include a method for assessing a sample from a subject for a coccidioidomycosis infection. In accordance with these embodiments, the method includes performing a reaction to detect the CTS1 detection agent bound to the recombinant CTS1 polypeptide in any of the compositions described herein, wherein anti-CTS1 antibodies present in the sample inhibit binding of the CTS1 detection agent to the recombinant CTS1 polypeptide, thereby indicating the presence of the anti-CTS1 antibodies in the sample.

Embodiments of the present disclosure also include a composition for detecting and/or quantifying Coccidioidal Chitinase-1 (CTS1) antigen. In accordance with these embodiments, the composition includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the composition further comprises a sample from a subject, wherein CTS1 present in the sample inhibits binding of the CTS1 detection agent to the recombinant CTS1 polypeptide, thereby facilitating detection of the CTS1 in the sample.

In some embodiments, the CTS1 detection agent comprises a CTS1 antibody or a CTS1 target antigen binding fragment thereof.

In some embodiments, the detection moiety comprises at least one of a peptide, a polypeptide, a fluorophore, a chromophore, a radiolabel, a nucleotide, a polynucleotide, a small molecule, an enzyme, and/or a nanoparticle.

In some embodiments, the sample is a serum sample from a subject that is infected with or suspected of being infected with Coccidioides spp.

In some embodiments, the recombinant CTS1 polypeptide is attached to a substrate.

In some embodiments, the composition further comprises a CTS1 calibrator or control polypeptide.

Embodiments of the present disclosure also include a method for assessing a sample from a subject for a coccidioidomycosis infection. In accordance with these embodiments, the method includes performing a reaction to detect the CTS1 detection agent bound to the recombinant CTS1 polypeptide in any of the compositions described herein, wherein CTS1 present in the sample inhibits binding of the CTS1 detection agent to the recombinant CTS1 polypeptide, thereby indicating the presence of CTS1 in the sample.

Embodiments of the present disclosure also include a composition for detecting and/or quantifying anti-Coccidioidal Chitinase-1 (CTS1) antibodies. In accordance with these embodiments, the composition includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the composition further comprises a sample from a subject, wherein anti-CTS1 antibodies present in the sample inhibit binding of the CTS1 detection agent to the recombinant CTS1 polypeptide, thereby facilitating detection of the anti-CTS1 antibodies in the sample.

In some embodiments, the CTS1 detection agent comprises a CTS1 antibody or a CTS1 target antigen binding fragment thereof.

In some embodiments, the detection moiety comprises at least one of a peptide, a polypeptide, a fluorophore, a chromophore, a radiolabel, a nucleotide, a polynucleotide, a small molecule, an enzyme, and/or a nanoparticle.

In some embodiments, the sample is a serum sample from a subject that is infected with or suspected of being infected with Coccidioides spp.

In some embodiments, the recombinant CTS1 polypeptide is attached to a substrate.

In some embodiments, the composition further comprises a CTS1 calibrator or control polypeptide.

Embodiments of the present disclosure also include a method for assessing a sample from a subject for a coccidioidomycosis infection, the method comprising: performing a reaction to detect the CTS1 detection agent bound to the recombinant CTS1 polypeptide in any of the compositions described herein, wherein anti-CTS1 antibodies present in the sample inhibit binding of the CTS1 detection agent to the recombinant CTS1 polypeptide, thereby indicating the presence of the anti-CTS1 antibodies in the sample.

Embodiments of the present disclosure also include a kit for detecting and/or quantifying Coccidioidal Chitinase-1 (CTS1) antigen and/or anti-CTS1 antibodies. In accordance with these embodiments, the kit includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the kit comprises a CTS1 calibrator or control polypeptide.

In some embodiments, the kit comprises instructions for performing an assay to detect and/or quantify Coccidioidal Chitinase-1 (CTS1) antigen and/or anti-CTS1 antibodies in a sample from a subject.

### BRIEF DESCRIPTION OF THE DRAWINGS

FIG. 1: Diagram of inhibition assay used for quantification of chitinase-1 (CTS1). Biontinylated monoclonal antibody 2F11 is incubated with patient serum at different dilutions for 1 hour. After incubation, mixture is added to an enzyme-linked immunosorbent assay (ELISA) plate that has been coated with recombinant CTS1 (rCTS1) and allowed to incubate. If CTS1 antigen and/or anti-CTS1 antibodies are present in the sample being tested, it will block the antibody from being able to bind rCTS1 in the ELISA plate, generating low signal. If CTS1 antigen and/or anti-CTS1 antibodies are not present, the antibody will bind rCTS1 in the ELISA plate, generating high signal. Schematic was created with BioRender.com. Abbreviations: CTS1, chitinase-1; ELISA, enzyme-linked immunosorbent assay; mAb, monoclonal antibody; SA-HRP, streptavidin-horseradish peroxidase.

FIGS. 2A-2B: FIG. 2A, Coomassie blue-stained sodium dodecyl-sulfate polyacrylamide gel electrophoresis of recombinant chitinase-1 (rCTS1) and PNGase F-treated rCTS1. FIG. 2B, Western blot probed with 2F11 monoclonal antibody.

FIG. 3: Quantification of chitinase-1 in different commercially available antigen preparations used in fungal diagnostics. Brackets at the bottom designate manufacturers of each antigen preparation. For values <0.155 μg/mL (the analytical limit of detection), the assigned value was zero. Abbreviations: Ag, antigen; CF, complement fixation; CTS1, chitinase-1; IDCF, immunodiffusion correspondent of complement fixation.

FIGS. 4A-4C: FIG. 4A, Composition of serum samples tested and sorted by diagnosis category. *Findings are defined as radiology findings and/or symptoms. **Serology is defined as positive complement fixation (CF), immunodiffusion (ID) (immunoglobulin G [IgG] or immunoglobulin M), or IgG by enzyme immunoassay. FIG. 4B, Calculated EIA units in all patient serum tested, separated by diagnostic category. Blue inverted triangles represent patients with proven diagnosis, while orange squares indicate patients with proven coccidioidomycosis (CM) whose disease appeared to be resolved at time of specimen collection (no symptoms and negative serology; see Table 1). A dotted line represents the cutoff value for positivity at 32.5 units, with any value above this categorized as a negative result (shaded area). FIG. 4C, Correlation of EIA units with serologic antibody titers determined by CF. Individual dots on the left side of the line show EIA units determined by enzyme-linked immunosorbent assay in 51 proven and probable patients with a positive CF titer (grouped by CF titer on x-axis). Individual dots on the right side of the line show EIA units in 27 proven and probable patients with a negative CF titer and either positive or negative ID results. Average EIA units by titer are represented with a line.

FIG. 5: Receiver operating characteristic plot for the diagnosis of coccidioidomycosis (CM) with use of the inhibition ELISA. Patients with proven and probable CM represent the true-positive group while patients categorized as not CM make up the true-negative group. Patients classified as possible CM were excluded from this analysis. The area under the curve was 0.9652 (standard error, 0.01441 [95% confidence interval, 0.9370-0.9929]; P<0.0001). With a cutoff of 32.5 EIA units, the sensitivity is 89.74% and the specificity is 94.90%.

FIG. 6: Sequence confirmation of recombinant CTS1 by mass spectrometry. Tryptic digest of recombinant CTS1 was analyzed by LC-MS/MS. Spectra were searched against a combined FASTA database of Coccidioides spp. proteomes (obtained from Uniprot) using Sequest in Proteome Discoverer v1.4.1.14 (Thermo). Endochitinase-1 (accession no. P0CB51) was identified with 82.44% coverage as seen in highlighted regions. The highlight color corresponds to peptide confidence, with green indicating high-confidence peptides and red indicating low-confidence peptides.

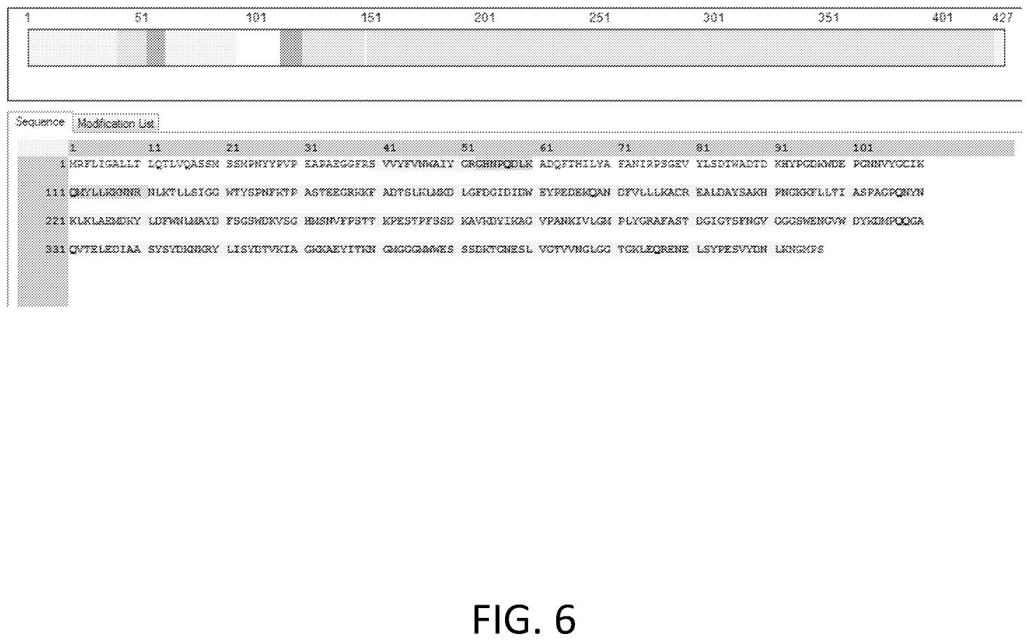

FIG. 7: Amino acid sequence alignments corresponding to nucleic acid sequences of SEQ ID NOs: 1-10.

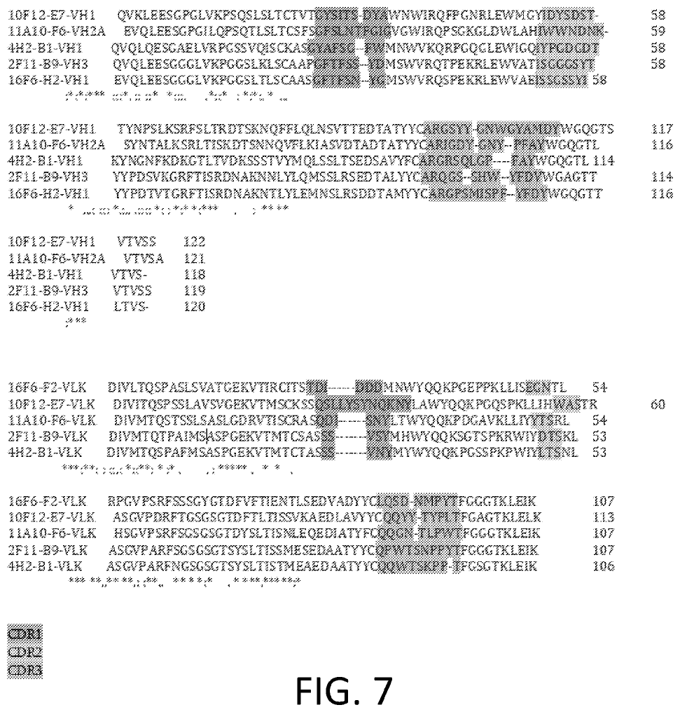

### DETAILED DESCRIPTION

Embodiments of present disclosure provide compositions and methods related to Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences for the production of a polypeptide, precursor, or RNA (e.g., rRNA, tRNA, sRNA, microRNA, lincRNA). The polypeptide can be encoded by a full-length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc.). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, a “double-stranded nucleic acid” may be a portion of a nucleic acid, a region of a longer nucleic acid, or an entire nucleic acid. A “double-stranded nucleic acid” may be, e.g., without limitation, a double-stranded DNA, a double-stranded RNA, a double-stranded DNA/RNA hybrid, etc. A single-stranded nucleic acid having secondary structure (e.g., base-paired secondary structure) and/or higher order structure comprises a “double-stranded nucleic acid”. For example, triplex structures are considered to be “double-stranded”. In some embodiments, any base-paired nucleic acid is a “double-stranded nucleic acid”

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA, including, but not limited to, oligos of peptide nucleic acids (PNA).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (the oligonucleotide or polynucleotide may be single-stranded), but may contain both the sense and anti-sense strands (the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample. The term “substantially purified” as used herein refers to a molecule such as a polypeptide, carbohydrate, nucleic acid etc. which is substantially free of other proteins, lipids, carbohydrates or other materials with which it is naturally associated. One skilled in the art can purify viral or bacterial polypeptides using standard techniques for protein purification. The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the viral or bacterial polypeptide can also be determined by amino-terminal amino acid sequence analysis. Other types of antigens such as polysaccharides, small molecule, mimics etc. are included within the present disclosure.

“Peptide” and “polypeptide” as used herein, and unless otherwise specified, refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein).

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

In some embodiments the substitutions can be conservative amino acid substitutions. Examples of conservative amino acid substitutions, unlikely to affect biological activity, include the following: alanine for serine, valine for isoleucine, aspartate for glutamate, threonine for serine, alanine for glycine, alanine for threonine, serine for asparagine, alanine for valine, serine for glycine, tyrosine for phenylalanine, alanine for proline, lysine for arginine, aspartate for asparagine, leucine for isoleucine, leucine for valine, alanine for glutamate, aspartate for glycine, and these changes in the reverse. See e.g., Neurath et al., The Proteins, Academic Press, New York (1979), the relevant portions of which are incorporated herein by reference. Further, an exchange of one amino acid within a group for another amino acid within the same group is a conservative substitution, where the groups are the following: (1) alanine, valine, leucine, isoleucine, methionine, norleucine, and phenylalanine: (2) histidine, arginine, lysine, glutamine, and asparagine; (3) aspartate and glutamate; (4) serine, threonine, alanine, tyrosine, phenylalanine, tryptophan, and cysteine; and (5) glycine, proline, and alanine.

The term “homology” and “homologous” refers to a degree of identity. There may be partial homology or complete homology. A partially homologous sequence is one that is less than 100% identical to another sequence.

As used herein, the terms “complementary” or “complementarity” are used in reference to polynucleotides (e.g., a sequence of nucleotides such as an oligonucleotide or a target nucleic acid) related by the base-pairing rules. For example, for the sequence “5′-A-G-T-3′“is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present disclosure and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

An “absolute amount” as used herein refers to the absolute value of a change or difference between at least two assay results taken or sampled at different time points and, which similar to a reference level, has been linked or is associated herein with various clinical parameters (e.g., presence of disease, stage of disease, severity of disease, progression, non-progression, or improvement of disease, etc.). “Absolute value” as used herein refers to the magnitude of a real number (such as, for example, the difference between two compared levels (such as levels taken at a first time point and levels taken at a second time point)) without regard to its sign, i.e., regardless of whether it is positive or negative.

This disclosure provides exemplary reference levels and absolute amounts (e.g., calculated by comparing reference levels at different time points). However, it is well-known that reference levels and absolute amounts may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels and absolute amounts for those other immunoassays based on the description provided by this disclosure. Whereas the precise value of the reference level and absolute amount may vary between assays, the findings as described herein should be generally applicable and capable of being extrapolated to other assays.

“Affinity matured antibody” is used herein to refer to an antibody with one or more alterations in one or more CDRs, which result in an improvement in the affinity (i.e., K_{D}, k_{d} or kₐ) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies is known in the art, including the screening of a combinatory antibody library that has been prepared using bio-display. For example, Marks et al., BioTechnology, 10: 779-783 (1992) describes affinity maturation by VH and VL domain shuffling. Random mutagenesis of CDR and/or framework residues is described by Barbas et al., Proc. Nat. Acad. Sci. USA, 91: 3809-3813 (1994); Schier et al., Gene, 169: 147-155 (1995); Yelton et al., J. Immunol., 155: 1994-2004 (1995); Jackson et al., J Immunol., 154(7): 3310-3319 (1995); and Hawkins et al, J. Mol. Biol., 226: 889-896 (1992).

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“CDR” is used herein to refer to the “complementarity determining region” within an antibody variable sequence. There are three CDRs in each of the variable regions of the heavy chain and the light chain. Proceeding from the N-terminus of a heavy or light chain, these regions are denoted “CDR1”, “CDR2”, and “CDR3”, for each of the variable regions. The term “CDR set” as used herein refers to a group of three CDRs that occur in a single variable region that binds the antigen. An antigen-binding site, therefore, may include six CDRs, comprising the CDR set from each of a heavy and a light chain variable region. A polypeptide comprising a single CDR, (e.g., a CDR1, CDR2, or CDR3) may be referred to as a “molecular recognition unit.” Crystallographic analyses of antigen-antibody complexes have demonstrated that the amino acid residues of CDRs form extensive contact with bound antigen, wherein the most extensive antigen contact is with the heavy chain CDR3. Thus, the molecular recognition units may be primarily responsible for the specificity of an antigen-binding site. In general, the CDR residues are directly and most substantially involved in influencing antigen binding.

The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as “Kabat CDRs”. Chothia and coworkers (Chothia and Lesk, J. Mol. Biol., 196: 901-917 (1987); and Chothia et al., Nature, 342: 877-883 (1989)) found that certain sub-portions within Kabat CDRs adopt nearly identical peptide backbone conformations, despite having great diversity at the level of amino acid sequence. These sub-portions were designated as “L1”, “L2”, and “L3”, or “H1”, “H2”, and “H3”, where the “L” and the “H” designate the light chain and the heavy chain regions, respectively. These regions may be referred to as “Chothia CDRs”, which have boundaries that overlap with Kabat CDRs. Other boundaries defining CDRs overlapping with the Kabat CDRs have been described by Padlan, FASEB J., 9: 133-139 (1995), and MacCallum, J. Mol. Biol., 262(5): 732-745 (1996). Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat- or Chothia-defined CDRs.

“Coefficient of variation” (CV), also known as “relative variability,” is equal to the standard deviation of a distribution divided by its mean.

“Component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, whole blood, serum or plasma sample, in accordance with the methods described herein and other methods known in the art. Some components can be in solution or lyophilized for reconstitution for use in an assay.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

The terms “administration of” and “administering” with respect to the compositions described herein generally refers to providing a composition of the present disclosure to a subject in need of treatment. For example, the composition can be administered (e.g., via injection) intravitreally (IVT), intracorneally, subconjunctivally, periocularly, suprachoroidally, intrasclerally, intracameally, or subretinally. Routes of systemic administration are also possible, in accordance with the compositions and methods described herein.

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier and/or excipient. When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

The term “pharmaceutical composition” as used herein refers to a composition that can be administered to a subject to treat or prevent a disease or pathological condition, and/or to improve/enhance one or more aspects of a subject's physical health. The compositions can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations containing pharmaceutically acceptable carriers are described in a number of sources which are well known and readily available to those skilled in the art. For example, Remington's Pharmaceutical Sciences (Martin E W, Remington's Pharmaceutical Sciences, Easton Pa., Mack Publishing Company, 19.sup.th ed., 1995) describes formulations that can be used in connection with the subject invention.

The term “pharmaceutically acceptable carrier, excipient, or vehicle” as used herein refers to a medium which does not interfere with the effectiveness or activity of an active ingredient and which is not toxic to the hosts to which it is administered and which is approved by a regulatory agency of the Federal or a state government or listed in the U.S. Pharmacopeia or other generally recognized pharmacopeia for use in animals, and more particularly in humans. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to saline, buffered saline, dextrose, water, glycerol, ethanol, and combinations thereof. The use of such media and agents for an active substance is well known in the art.

As used herein, the term “effective amount” generally means that amount of a drug or pharmaceutical agent that will elicit the biological or medical response of a tissue, system, animal or human that is being sought, for instance, by a researcher or clinician. Furthermore, the term “therapeutically effective amount” generally means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The term “combination” and derivatives thereof, as used herein, generally means either, simultaneous administration or any manner of separate sequential administration of a therapeutically effective amount of Compound A, or a pharmaceutically acceptable salt thereof, and Compound B or a pharmaceutically acceptable salt thereof, in the same composition or different compositions. If the administration is not simultaneous, the compounds are administered in a close time proximity to each other. Furthermore, it does not matter if the compounds are administered in the same dosage form (e.g., one compound may be administered topically and the other compound may be administered orally).

As used herein, the term “subject” and “patient” as used herein interchangeably refers to any vertebrate, including, but not limited to, cow, pig, camel, llama, horse, donkey, mule, zebra, goat, rabbit, sheep, hamsters, guinea pig, cat, dog, rat, mouse, non-human primates, and humans. In some embodiments, the subject may be an equid, which refers to any species from the genus Equus, including but not limited to, horses, donkeys, zebras, and mules, and any variant thereof. The subject or patient may be undergoing various forms of treatment separate and independent of the methods described herein.

As used herein, the term “treat,” “treating” or “treatment” are each used interchangeably herein to describe reversing, alleviating, or inhibiting the progress of a disease and/or injury, or one or more symptoms of such disease, to which such term applies, and/or to improve/enhance one or more aspects of a subject's physical health. Depending on the condition of the subject, the term also refers to preventing a disease, and includes preventing the onset of a disease, or preventing the symptoms associated with a disease (e.g., coccidioidomycosis). A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

As used herein, the term “salts” and “pharmaceutically acceptable salts” generally refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quatemary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disuSfonic oxalic, and isethionic, and the like. Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, isopropanol, and the like. Lists of suitable salts can be found, for example, in Remington's Pharmaceutical Sciences, 17th ed., Mack Publishing Company, Easton, Pa., 985.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.) This disclosure provides exemplary reference levels. However, it is well-known that reference levels may vary depending on the nature of the immunoassay (e.g., antibodies employed, reaction conditions, sample purity, etc.) and that assays can be compared and standardized. It further is well within the ordinary skill of one in the art to adapt the disclosure herein for other immunoassays to obtain immunoassay-specific reference levels for those other immunoassays based on the description provided by this disclosure. Whereas the precise value of the reference level may vary between assays, the findings as described herein should be generally applicable and capable of being extrapolated to other assays.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a reference level or control level (e.g., “low”, “medium”, or “high” levels), can be used. Multiple calibrators (i.e., more than one calibrator or a varying amount of calibrator(s)) can be used in conjunction to comprise a “sensitivity panel.”

“Detection moiety” and “detectable label” as used herein refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromogens, fluorescent compounds, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, may not be detectable but may become detectable upon reaction with yet another moiety. Use of the term “detectably labeled” is intended to encompass such labeling. In some embodiments, the term “detection moiety” is any moiety or compound that is detectable by methods including, but not limited to, spectroscopic, photochemical, biochemical, immunochemical, chemical, electrochemical, radioactivity, and other physical means. A detection moiety may be detectable indirectly; for example, the detection moiety may be a moiety or compound that is a member of a specific binding pair, wherein the second member of the binding pair includes a detection moiety that can be detected directly. A non-limiting and known example of such a detection moiety is biotin, which may bind to avidin or streptavidin comprising a detection moiety such as a fluorophore.

Exemplary detection moieties include, but are not limited to, fluorophores, chromophores, radiolabels, polynucleotides, small molecules, enzymes, nanoparticles, and upconverters.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e., a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Linearity” refers to how well the method or assay's actual performance across a specified operating range approximates a straight line. Linearity can be measured in terms of a deviation, or non-linearity, from an ideal straight line. “Deviations from linearity” can be expressed in terms of percent of full scale. In some of the methods disclosed herein, less than 10% deviation from linearity (DL) is achieved over the dynamic range of the assay. “Linear” means that there is less than or equal to about 20%, about 19%, about 18%, about 17%, about 16%, about 15%, about 14%, about 13%, about 12%, about 11%, about 10%, about 9%, or about 8% variation for or over an exemplary range or value recited.

“Substrate” as used herein refers to a variety of different types of surfaces or structures that allow for the application of one or more detection agents or recombinant polypeptides. In some embodiments, the substrate is a biosensor, an assay plate, or the like. For example, the substrate may be an optical biosensor, such as those described in U.S. Pat. Nos. 5,313,264, 5,846,842, 5,496,701, etc. The substrate may also be a potentiometric or electrochemical biosensor, such as described in U.S. Pat. No. 5,413,690, or PCT Application WO98/35232. The substrate may be a diamond film biosensor, such as described in U.S. Pat. No. 5,777,372. Accordingly, the substrate may be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. For example, the substrate may comprise polyethylene, polytetrafluoroethylene, polystyrene, polyethylene terephthalate, polycarbonate, gold, silicon, silicon oxide, silicon oxynitride, indium, tantalum oxide, niobium oxide, titanium, titanium oxide, platinum, iridium, indium tin oxide, diamond or diamond-like film, etc. The substrate may be a substrate suitable for “chip-based” and “pin-based” combinatorial chemistry techniques. All can be prepared in accordance with known techniques. See, e.g., U.S. Pat. Nos. 5,445,934, 5,288,514 and 5,624,711. Substrates as described above can be formed of any suitable material, including but not limited to a material selected from the group consisting of metals, metal oxides, alloys, semiconductors, polymers (such as organic polymers in any suitable form including woven, nonwoven, molded, extruded, cast, etc.), silicon, silicon oxide, ceramics, glass, and composites thereof.

Polymers used to form substrates as described herein may be any suitable polymer, including but not limited to: poly(ethylene) (PE), poly(propylene) (PP), cis and trans isomers of poly(butadiene) (PB), cis and trans isomers of poly(isoprene), poly(ethylene terephthalate) (PET), polystyrene (PS), polycarbonate (PC), poly(epsilon-caprolactone) (PECL or PCL), poly(methyl methacrylate) (PMMA) and its homologs, poly(methyl acrylate) and its homologs, poly(lactic acid) (PLA), poly(glycolic acid), polyorthoesters, poly(anhydrides), nylon, polyimides, polydimethylsiloxane (PDMS), polybutadiene (PB), polyvinylalcohol (PVA), polyacrylamide and its homologs such as poly(N-isopropyl acrylamide), fluorinated polyacrylate (PFOA), poly(ethylene-butylene) (PEB), polystyrene-acrylonitrile) (SAN), polytetrafluoroethylene (PTFE) and its derivatives, polyolefin plastomers, and combinations and copolymers thereof, etc. The substrate may optionally have an additional layer such as a gold or an oxide layer formed on the surface of the substrate, for example, to facilitate the deposition of a polymer layer or a linking layer, as discussed further below.

### 2. COMPOSITIONS AND METHODS

Current diagnostic methods for coccidioidomycosis rely on a constellation of clinical, radiologic, and laboratory findings that may be conflicting, and in the case of serodiagnostics may be inconsistent. However, their collective use is the current accepted standard used to inform clinical decisions, and often repeated testing over time is needed to clarify patient status. Further complicating diagnosis is the possibility that early treatment of coccidioidomycosis may abrogate seroconversion, which can make definitive diagnosis nearly impossible in some patients with other comorbidities that have overlapping symptoms. Another group did not report this phenomenon. Although a comparison cannot be made between the two studies, the different experience of each group highlights the challenges of antibody-based methods as a component of diagnosis. Yet, various CTS1 antigen preparations have been used in antibody-based diagnostic assays for over six decades. Although CTS1 may be an accepted serologic target, antibody responses in individuals infected with Coccidioides spp are often delayed or even absent, especially in immunocompromised patients. However, if a fungal antigen could be detected in a biofluid, a more definitive diagnosis could be made, independent of the host immune response.

Complexity and inconsistency in the current diagnostic testing regimen make evaluation of a new diagnostic tool challenging. Several groups have proposed criteria to classify patients with varying certainties of coccidioidomycosis infection to aid with this challenge. The patients included in this study were categorized using the general definitions offered by the EORTC and the MSG, which provide criteria for proven, probable, and not coccidioidomycosis. The remaining patients who did not fit these criteria were categorized as possible coccidioidomycosis and were excluded from the sensitivity and specificity analysis since their true status is unknown. The results show that the use of the EIA provides sensitivity and specificity comparable only to the use of multiple existing serodiagnostic tests. While the assay measures CTS1 antigen, potential interference from anti-CTS1 antibodies in patient sera may interfere with absolute CTS1 antigen quantification. Nonetheless, the assay was positive for 4 sera from patients who had negative CF and ID results, demonstrating the value of the assay in detecting active infection. The updated results suggest that, for human specimens, the assay yields a positive result from either CTS1 antigen or antibodies against CTS1, providing a distinct advantage over other assays. This feature was borne out in the clinical performance of the assay, whereby this assay alone was as good as the composite clinical standard, and better than any other single serologic approach. Some patients take many weeks to become antibody positive using current tests and are given 1 or more courses of unnecessary antibiotics, posing risk for patients and contributing to antimicrobial resistance.

Another use of this assay is to measure the CTS1 present in antigen preparations, and potential lot-to-lot variability. While the relative amounts of CTS1 between commercial products is somewhat irrelevant, it is essential to have consistent reagents for diagnostic methods. Importantly, Histoplasma and Biastomyces antigen ID preparations from IMMY did not cross-react on the assay, demonstrating a critical feature of analytical specificity of the assay. Nine patients had other fungal infections and were negative by the assay, suggesting that the assay has good clinical specificity. More samples from patients with other fungal infections will need to be tested to further characterize clinical specificity of the assay.

The samples tested in this study are a single point in time from individuals at different stages of disease with a wide range in time since diagnosis, time since treatment initiation, and/or time since symptom resolution. In some cases, the course of disease led to evidence that would categorize the patient as a proven case. Patient samples were categorized based on information available at the time of blood draw. Additional context from future events is available in Table 1 for 30 patients.

TABLE 1
Additional information on 30 samples. Samples in this table include those with proven and probable coccidioidomycosis
(CM) who were below the cutoff for positivity or were categorized as probable but later would have been proven;
all samples in the possible CM category; and patients classified as not CM who had a positive result.

| | | | | | | | | SUBSEQUENT | |
|---|---|---|---|---|---|---|---|---|---|
| | HISTORY, | | | SEROLOGICAL TEST | | | EIA | DISEASE | LIKELY BEST-FIT |
| PATIENT | PRESENTATION | Category | RADIOLOGY ^{#} | CF | ID | EIA | UNITS | COURSE | CLASSIFICATION |
| 1 | Dx with proven | Proven | Nodule with | Neg | IgG− | IgG in. | 34.48 | | Resolving/ |
| | CM 12 months | | stable | | IgM− | IgM− | | | Controlled |
| | prior to specimen | | surrounding | | | | | | |
| | collection. | | micronodules | | | | | | |
| | Subsequent | | | | | | | | |
| | antifungal tx. | | | | | | | | |
| | No symptoms | | | | | | | | |
| | reported at time | | | | | | | | |
| | of specimen | | | | | | | | |
| | collection | | | | | | | | |
| 2 | Cough, dyspnea at | Probable | Innumerable | 2 | IgG+ | IgG+ | 9.13 | 11 months later | Chronic |
| | time of specimen | | small | | IgM+ | IgM− | | culture and | |
| | collection | | pulmonary | | | | | PCR positive | |
| | | | nodules | | | | | for Coccidioides | |
| 3 | Feels ill, feverish, | Probable | 1 month after | Neg | IgG− | IgG+ | 42.55 | Blood culture | Resolving/ |
| | general malaise | | specimen | | IgM + | IgM− | | positive for | Controlled |
| | | | collection | | | | | Candida. 4 | |
| | | | shows nodule | | | | | months later | |
| | | | with ground | | | | | negative by ID | |
| | | | glass opacities | | | | | | |
| 4 | Severe fatigue, | Probable | Lungs appear | Neg | IgG− | IgG+ | 33.65 | Remained IgG+ | Resolving/ |
| | shortness of | | clear. X-ray 6 | | IgM− | IgM− | | by EIA 5 | Controlled |
| | breath. Symptoms | | months prior | | | | | months after | |
| | have been present | | shows two | | | | | specimen | |
| | for 6 months | | nodules. | | | | | collection | |
| 5 | Dx with CAP | Probable | 1 cm nodular | Neg | IgG− | IgG+ | 32.7 | No future | Resolving/ |
| | suggestive of CM | | density | | IgM− | IgM+ | | serology | Controlled |
| | 9 months prior to | | | | | | | performed | |
| | specimen collection. | | | | | | | | |
| | Deep cough with | | | | | | | | |
| | yellow-green sputum | | | | | | | | |
| | and shortness of | | | | | | | | |
| | breath at time of | | | | | | | | |
| | specimen collection. | | | | | | | | |
| | Family came down | | | | | | | | |
| | with similar acute | | | | | | | | |
| | respiratory illness | | | | | | | | |
| | after international | | | | | | | | |
| | travel but have | | | | | | | | |
| | recovered. | | | | | | | | |
| 6 | Hx of end-stage | Probable | Right | Neg | IgG− | IgG+ | 35.95 | No future | Undetermined |
| | renal disease. | | peripheral | | IgM− | IgM− | | serology | |
| | Hospitalized 2-3 | | lung nodule. | | | | | performed | |
| | months prior to | | Clustered | | | | | | |
| | specimen | | nodularity | | | | | | |
| | collection for | | in left lung | | | | | | |
| | bacteremia, no | | apex, some | | | | | | |
| | incidences since | | tree-in-bud | | | | | | |
| | then. | | configuration | | | | | | |
| 7 | Kidney transplant | Probable | Calcified | Neg | IgG− | IgG+ | 41.04 | Subsequent | Resolving/ |
| | evaluation 9 | | granuloma, | | IgM+ | IgM− | | serology | Controlled |
| | months prior to | | numerous tiny | | | | | remains the | |
| | specimen | | nodules appear | | | | | same | |
| | collection, positive | | partially calcified. | | | | | | |
| | for CM IgM by ID | | | | | | | | |
| | and IgG by EIA. | | | | | | | | |
| | No hx of | | | | | | | | |
| | symptomatic CM. | | | | | | | | |
| | Asymptomatic at | | | | | | | | |
| | time of specimen | | | | | | | | |
| | collection | | | | | | | | |
| 8 | Hx of AML. | Probable | Multifocal | Neg | IgG− | IgG+ | 61.6 | Remained IgM+ | Resolving/ |
| | Hospitalized 1 | | ground-glass | | IgM+ | IgM− | | by ID one | Controlled |
| | month prior to | | opacities and | | | | | month later, | |
| | specimen | | consolidative | | | | | then negative 3 | |
| | collection due to | | nodularities | | | | | months later | |
| | fatigue and | | significantly | | | | | | |
| | weakness. | | improved | | | | | | |
| | Antifungal tx | | | | | | | | |
| | initiated due to | | | | | | | | |
| | positive IgG by | | | | | | | | |
| | EIA. Reports | | | | | | | | |
| | fatigue, weakness | | | | | | | | |
| | and mild cough at | | | | | | | | |
| | time of specimen | | | | | | | | |
| | collection | | | | | | | | |
| 9 | Went to urgent | Probable | Focal | Neg | IgG− | IgG+ | 41.18 | IgG+ by ID 1 | Potentially Acute |
| | care 2 weeks prior | | consolidation | | IgM+ | IgM+ | | month later. | infection |
| | to specimen | | consistent | | | | | Negative by ID | |
| | collection, | | with left lower | | | | | but IgG+ by | |
| | initiated on 10-day | | lobe pneumonia | | | | | EIA 3 months | |
| | course of | | | | | | | later. CF titer | |
| | Levaquin, then | | | | | | | positive at 1:2 | |
| | Cefuroxime due to | | | | | | | 6 months later | |
| | failure to resolve | | | | | | | with subsequent | |
| | symptoms. At time | | | | | | | increase to 1:4 | |
| | of specimen | | | | | | | | |
| | collection is | | | | | | | | |
| | having fevers, | | | | | | | | |
| | night sweats, sinus | | | | | | | | |
| | congestion, cough, | | | | | | | | |
| | and chest tightness | | | | | | | | |
| 10 | Hx of CM by | Possible | Mild bibasilar | Neg | IgG− | IgG+ | 46.08 | Subsequent | Resolving/ |
| | positive serology | | subsegmental | | IgM eq. | IgM− | | serology since | Controlled |
| | 13 months prior to | | atelectasis | | | | | transplant has | |
| | specimen collection, | | | | | | | been | |
| | antifungal tx | | | | | | | consistently | |
| | initiated. At time | | | | | | | negative | |
| | of specimen | | | | | | | | |
| | collection patient | | | | | | | | |
| | undergoing liver | | | | | | | | |
| | transplant, | | | | | | | | |
| | antifungal tx | | | | | | | | |
| | resumed. | | | | | | | | |
| 11 | Symptoms 1 | Possible | Nodular mass- | Neg | IgG− | IgG− | 31.99 | IgG+ by EIA | Resolving/ |
| | month prior | | like consolidation | | IgM eq. | IgM− | | only 5 months | Controlled |
| | included profound | | with surrounding | | | | | later. Serology | |
| | fatigue, severe | | ground-glass | | | | | since then has | |
| | headaches, | | and numerous | | | | | been negative. | |
| | palpitations, chest | | satellite nodules | | | | | | |
| | pain, malaise, | | | | | | | | |
| | night sweats. At | | | | | | | | |
| | time of specimen | | | | | | | | |
| | collection patient | | | | | | | | |
| | reports improvement, | | | | | | | | |
| | has completed | | | | | | | | |
| | 2 weeks of | | | | | | | | |
| | antifungal tx. | | | | | | | | |
| 12 | Presented 3-4 | Possible | No | Neg | IgG− | IgG− | 47.73 | Continued IgM | Undetermined |
| | months prior to | | abnormalities | | IgM− | IgM in. | | in. or + result | |
| | specimen | | noted | | | | | by EIA only. | |
| | collection for | | | | | | | CF and ID | |
| | cough, extreme | | | | | | | consistently | |
| | fatigue, sore | | | | | | | negative. | |
| | throat, shortness of | | | | | | | | |
| | breath. Initiated | | | | | | | | |
| | antifungal tx and | | | | | | | | |
| | noted slow | | | | | | | | |
| | improvement. | | | | | | | | |
| | Stopped antifungal | | | | | | | | |
| | tx two weeks prior | | | | | | | | |
| | to specimen | | | | | | | | |
| | collection, noticed | | | | | | | | |
| | worsening of chest | | | | | | | | |
| | discomfort and | | | | | | | | |
| | fatigue. | | | | | | | | |
| 13 | No clinical notes | Possible | 2 nodular | Neg | N/A | IgG− | 29.36 | No previous or | Undetermined |
| | about symptoms at | | opacities, both | | | IgM− | | subsequent | |
| | time of specimen | | noncalcified | | | | | serology | |
| | collection. | | | | | | | | |
| 14 | Dx with CM 18 | Possible | Chest x-ray 6 | Neg | IgG− | IgG− | 41.33 | | Resolving/ |
| | months prior to | | months prior | | IgM− | IgM in. | | | Controlled |
| | specimen | | to specimen | | | | | | |
| | collection due to | | collection shows | | | | | | |
| | symptoms and | | decrease in | | | | | | |
| | serology. At time | | size and | | | | | | |
| | of specimen | | density of | | | | | | |
| | collection patient | | nodule, no | | | | | | |
| | has completed 18 | | new nodules | | | | | | |
| | months of | | noted | | | | | | |
| | antifungal tx and | | | | | | | | |
| | is asymptomatic | | | | | | | | |
| 15 | Dx with CAP 2 | Possible | Chest CT 2 | Neg | IgG− | IgG− | 27.29 | All future | Resolving/ |
| | years prior to | | months prior | | IgM− | IgM− | | serology | Controlled |
| | specimen | | to specimen | | | | | remains | |
| | collection, | | collection shows | | | | | negative | |
| | resolved. Dx. With | | improvement | | | | | | |
| | probable CM 1 | | in tree-in-bud | | | | | | |
| | year prior to | | nodules. Chest | | | | | | |
| | specimen | | x-ray 1 month | | | | | | |
| | collection due to | | after specimen | | | | | | |
| | fatigue, fever, | | collection shows | | | | | | |
| | cough, shortness | | peribronchial | | | | | | |
| | of breath. | | vascular nodules. | | | | | | |
| | Antifungal tx for | | | | | | | | |
| | 4-6 weeks | | | | | | | | |
| | followed by | | | | | | | | |
| | resolution of | | | | | | | | |
| | symptoms. At time | | | | | | | | |
| | of specimen | | | | | | | | |
| | collection has | | | | | | | | |
| | experienced a | | | | | | | | |
| | relapse of | | | | | | | | |
| | symptoms over | | | | | | | | |
| | past two months. | | | | | | | | |
| 16 | Symptoms | Possible | Calcified | Neg | IgG− | IgG− | 58.84 | No subsequent | Undetermined |
| | unclear/not | | granuloma | | IgM− | IgM− | | serology | |
| | specified at time | | right middle | | | | | | |
| | of specimen | | lobe; nodule | | | | | | |
| | collection. Within | | right lower | | | | | | |
| | 2 years prior to | | lobe | | | | | | |
| | specimen | | | | | | | | |
| | collection has | | | | | | | | |
| | been dx with lupus | | | | | | | | |
| | erythematosus, | | | | | | | | |
| | babesia, | | | | | | | | |
| | toxoplasma, and | | | | | | | | |
| | chlamydia | | | | | | | | |
| | pneumonia | | | | | | | | |
| 17 | Tx with | Possible | No clear | Neg | IgG− | IgG+ | 21.15 | No subsequent | Resolving/ |
| | azithromycin and | | abnormalities | | IgM− | IgM− | | serology | Controlled |
| | prednisone 7 | | noted | | | | | | |
| | months prior to | | | | | | | | |
| | specimen | | | | | | | | |
| | collection for | | | | | | | | |
| | pneumonia. 6 | | | | | | | | |
| | months prior was | | | | | | | | |
| | dx with CM by | | | | | | | | |
| | positive serology | | | | | | | | |
| | but elected not to | | | | | | | | |
| | initiate antifungal | | | | | | | | |
| | tx. Specimen | | | | | | | | |
| | collected one | | | | | | | | |
| | month post sx, | | | | | | | | |
| | feels somewhat | | | | | | | | |
| | sub-fatigued | | | | | | | | |
| 18 | Dx with possible | Possible | Lungs appear | 2 | IgG+ | IgG− | 16.53 | CF titer | Resolving/ |
| | CM 6 months | | clear | | IgM− | IgM in. | | remained at 1:2 | Chronic |
| | prior to specimen | | | | | | | one year after | |
| | collection. | | | | | | | specimen | |
| | Asymptomatic at | | | | | | | collection | |
| | time of collection. | | | | | | | | |
| 19 | Slightly increased | Possible | 6 months prior | Neg | IgG− | IgG− | 44.87 | No subsequent | Undetermined |
| | fatigue, upper | | to specimen | | IgM− | IgM+ | | serology | |
| | respiratory | | collection no | | | | | | |
| | symptoms, | | abnormalities | | | | | | |
| | allergies, | | noted. 2 | | | | | | |
| | inconsistent cough | | weeks post | | | | | | |
| | and asthma. | | specimen | | | | | | |
| | Patient concerned | | collection | | | | | | |
| | about a neck lump | | chest CT notes | | | | | | |
| | that has increased | | 2 mm nodule | | | | | | |
| | in size over past 3 | | “probably of | | | | | | |
| | months. | | no clinical | | | | | | |
| | | | consequence” | | | | | | |
| 20 | Dx with CM 20 | Possible | Lungs appear | 2 | IgG+ | IgG− | 21.33 | Subsequent CF | Resolving/ |
| | months prior to | | clear | | IgM− | IgM in. | | titer negative | Controlled |
| | specimen | | | | | | | but remained | |
| | collection. Over | | | | | | | IgG+ by ID | |
| | 12 months of | | | | | | | | |
| | antifungal tx. | | | | | | | | |
| | Asymptomatic at | | | | | | | | |
| | time of specimen | | | | | | | | |
| | collection | | | | | | | | |
| 21 | Dx with acute | Possible | No | Neg | IgG− | IgG− | 38.34 | Subsequent | Undetermined |
| | sinusitis one week | | abnormalities | | IgM− | IgM+ | | serology | |
| | prior to specimen | | noted | | | | | remains IgM+ | |
| | collection, tx with | | | | | | | by EIA | |
| | Augmentin. At | | | | | | | | |
| | time of specimen | | | | | | | | |
| | collection sinus | | | | | | | | |
| | pressure has | | | | | | | | |
| | resolved but has | | | | | | | | |
| | lingering | | | | | | | | |
| | headache, sweats, | | | | | | | | |
| | hot flashes, sore | | | | | | | | |
| | throat. | | | | | | | | |
| 22 | Subtle medial | Possible | 19 mm | Neg | N/A | IgG− | 22.86 | Subsequent | Undetermined |
| | subclavicular | | indeterminate | | | IgM− | | serology | |
| | swelling, | | nodule, not | | | | | includes EIA | |
| | asymptomatic | | definitively | | | | | only, remained | |
| | | | identified on | | | | | negative | |
| | | | lateral image. | | | | | | |
| | | | No prior | | | | | | |
| | | | imaging for | | | | | | |
| | | | comparison. | | | | | | |
| | | | Lungs are | | | | | | |
| | | | otherwise | | | | | | |
| | | | clear. | | | | | | |
| 23 | Dx with | Possible | Progression | Neg | IgG− | IgG in. | 49.52 | Serology by | Undetermined |
| | sarcoidosis 5 | | parabronchial | | IgM− | IgM in. | | EIA sent out | |
| | months prior to | | mediastinal | | | | | one week later | |
| | specimen | | lymph node | | | | | IgG− and IgM− | |
| | collection, treated | | enlargement | | | | | | |
| | with steroids but | | consistent | | | | | | |
| | had return of | | with treated or | | | | | | |
| | symptoms with | | resolving | | | | | | |
| | attempt to | | sarcoidosis | | | | | | |
| | decrease steroid | | | | | | | | |
| | dose (chest pain, | | | | | | | | |
| | dry cough). | | | | | | | | |
| 24 | Hx of CM 5 years | Possible | Left perihilar | Neg | N/A | IgG− | 20.21 | No subsequent | Resolving/ |
| | prior to specimen | | lesion stable | | | IgM− | | serology | Chronic |
| | collection, also hx | | since 2017 | | | | | | |
| | of COPD. At time | | | | | | | | |
| | of specimen | | | | | | | | |
| | collection has | | | | | | | | |
| | cough, occasional | | | | | | | | |
| | wheezing, oral | | | | | | | | |
| | lesion on lip. | | | | | | | | |
| 25 | Patient being | Possible | Scattered sub | Neg | IgG− | IgG− | 21.98 | | Undetermined |
| | evaluated post- | | centimeter | | IgM− | IgM− | | | |
| | transplant | | pulmonary | | | | | | |
| | | | nodules stable | | | | | | |
| | | | from 2016. | | | | | | |
| | | | Some | | | | | | |
| | | | atelectasis | | | | | | |
| 26 | Patient being | Not CM | No | Neg | N/A | IgG− | 25.90 | Subsequent | Undetermined |
| | evaluated for | | abnormalities | | | IgM− | | serology | |
| | transplant | | noted | | | | | negative. | |
| 27 | Patient being | Not CM | | Neg | N/A | IgG− | 28.41 | | Undetermined |
| | evaluated for | | | | | IgM− | | | |
| | transplant | | | | | | | | |
| 28 | Patient being | Not CM | No | Neg | N/A | IgG− | 27.09 | | Undetermined |
| | evaluated for | | abnormalities | | | IgM− | | | |
| | transplant | | noted | | | | | | |
| 29 | Patient being | Not CM | No | Neg | N/A | IgG− | 25.85 | | Undetermined |
| | evaluated for | | abnormalities | | | IgM− | | | |
| | kidney donation | | noted | | | | | | |
| 30 | Hx of RA. Patient | Not CM | No | Neg | IgG− | IgG− | 24.41 | Subsequent | Undetermined |
| | reports hot flashes | | abnormalities | | IgM− | IgM− | | serology | |
| | and night sweats | | noted | | | | | continues to be | |
| | for past month. | | | | | | | negative. | |

^{#} radiology was within 3 months of specimen collection unless otherwise noted.
in = indeterminate
eq = equivocal
CM = coccidioidomycosis
DCM = disseminated coccidioidomycosis
Dx = diagnosed
Hx = history
Tx = treatment

In one example, patient 3 was characterized as probable due to innumerable pulmonary nodules and positive serology by CF (1:2), ID, and EIA. The patient's serum was quantified at 9.13 EIA units at that time. The patient was initiated on antifuingal treatment 4-5 weeks later but 11 months later returned a positive PCR, confirming proven infection. It would be difficult to know whether this patient was sufficiently treated, given this presentation. Using the CTS1 EIA in this report, trending of antigenemia and/or seroreactivity may correlate with clinical response and/or response to treatment. This assay is more likely to be sensitive in early infection, detecting antigen, as well as sustaining sensitivity for active disease by also detecting antibody.

The one other antigen assay described for Coccidioides was recently shown to be positive in only 38.6% of sera from immunocompetent patients and only 37.1% of pulmonary cases. That assay is also positive more often in disseminated cases, which typically have other indicators of infection. The CTS1 detection assay reported here has the potential for similar or better performance vs serologic assays.

In accordance with the above description, embodiments of the present disclosure include a composition detecting and/or quantifying Coccidioidal Chitinase-1 (CTS1) antigen and/or anti-CTS1 antibodies. In accordance with these embodiments, the composition includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the composition further comprises a sample from a subject, wherein: (a) CTS1 present in the sample inhibits binding of the CTS1 detection agent to the recombinant CTS1 polypeptide, thereby facilitating detection of the CTS1 in the sample; and/or (b) anti-CTS1 antibodies present in the sample inhibit binding of the CTS1 detection agent to the recombinant CTS1 polypeptide, thereby facilitating detection of the anti-CTS1 antibodies in the sample.

In some embodiments, the CTS1 detection agent comprises a CTS1 antibody or a CTS1 target antigen binding fragment thereof.

In some embodiments, the detection moiety comprises at least one of a peptide, a polypeptide, a fluorophore, a chromophore, a radiolabel, a nucleotide, a polynucleotide, a small molecule, an enzyme, and/or a nanoparticle. In some embodiments, the small molecule is biotin. In some embodiments, the polypeptide is streptavidin.

In some embodiments, the sample is a serum sample from a subject that is infected with or suspected of being infected with Coccidioides spp.

In some embodiments, the recombinant CTS1 polypeptide is attached to a substrate. In some embodiments, the substrate comprises glass, silicon, a metal oxide, and/or a polymer.

In some embodiments, the composition further comprises a CTS1 calibrator or control polypeptide.

Embodiments of the present disclosure also include a composition for detecting and/or quantifying Coccidioidal Chitinase-1 (CTS1) antigen. In accordance with these embodiments, the composition includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the composition further comprises a sample from a subject, wherein CTS1 present in the sample inhibits binding of the CTS1 detection agent to the recombinant CTS1 polypeptide, thereby facilitating detection of the CTS1 in the sample.

In some embodiments, the CTS1 detection agent comprises a CTS1 antibody or a CTS1 target antigen binding fragment thereof.

In some embodiments, the detection moiety comprises at least one of a peptide, a polypeptide, a fluorophore, a chromophore, a radiolabel, a nucleotide, a polynucleotide, a small molecule, an enzyme, and/or a nanoparticle.

In some embodiments, the sample is a serum sample from a subject that is infected with or suspected of being infected with Coccidioides spp.

In some embodiments, the recombinant CTS1 polypeptide is attached to a substrate.

In some embodiments, the composition further comprises a CTS1 calibrator or control polypeptide.

Embodiments of the present disclosure also include a composition for detecting and/or quantifying anti-Coccidioidal Chitinase-1 (CTS1) antibodies. In accordance with these embodiments, the composition includes a CTS1 detection agent comprising a CTS1 target antigen binding domain and a detection moiety; and a recombinant CTS1 polypeptide comprising a CTS1 target antigen detectable by the CTS1 detection agent.

In some embodiments, the composition further comprises a sample from a subject, wherein anti-CTS1 antibodies present in the sample inhibit binding of the CTS1 detection agent to the recombinant CTS1 polypeptide, thereby facilitating detection of the anti-CTS1 antibodies in the sample.

In some embodiments, the CTS1 detection agent comprises a CTS1 antibody or a CTS1 target antigen binding fragment thereof.

In some embodiments, the detection moiety comprises at least one of a peptide, a polypeptide, a fluorophore, a chromophore, a radiolabel, a nucleotide, a polynucleotide, a small molecule, an enzyme, and/or a nanoparticle.

In some embodiments, the sample is a serum sample from a subject that is infected with or suspected of being infected with Coccidioides spp.

In some embodiments, the recombinant CTS1 polypeptide is attached to a substrate.

In some embodiments, the composition further comprises a CTS1 calibrator or control polypeptide.

Embodiments of the present disclosure also include a kit for detecting and/or quantifying Coccidioidal Chitinase-1 (CTS1) antigen and/or anti-CTS1 antibodies. In accordance with these embodiments, the kit includes a CTS1 detection agent comprising a CTS1 target antigen binding domain and a detection moiety, and a recombinant CTS1 polypeptide comprising a CTS1 target antigen detectable by the CTS1 detection agent, as described further herein.

In some embodiments, the kit comprises a CTS1 calibrator or control polypeptide. In some embodiments, the kit comprises instructions for performing an assay to detect and/or quantify Coccidioidal Chitinase-1 (CTS1) antigen and/or anti-CTS1 antibodies in a sample from a subject.

In accordance with these embodiments, kits of the present disclosure can be used for assaying or assessing a test sample for coccidioidal CTS1 antigen and/or anti-CTS1 antibodies in human serum, including fragments thereof. The kit can comprise instructions for assaying the test sample for a CTS1 antigen and/or anti-CTS1 antibodies by immunoassay (e.g., chemiluminescent microparticle immunoassay). Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The kit may also include at least one composition comprising one or more isolated antibodies or antibody fragments thereof that specifically bind to a CTS1 antigen. The antibody may be a CTS1 detection antibody and/or capture antibody. Alternatively or additionally, the kit can comprise a calibrator or control (e.g., purified, and optionally lyophilized, CTSI antigen) and/or at least one container (e.g., tube, microtiter plates or strips, which can be already coated with a CTS1 antigen and/or a CTS1 antibody) for conducting the assay, and/or a buffer, such as an assay buffer or a wash buffer, either one of which can be provided as a concentrated solution, a substrate solution for the detectable label (e.g., an enzymatic label), or a stop solution. Preferably, the kit comprises all components, i.e., reagents, standards, buffers, diluents, etc., which are necessary to perform the assay. The instructions also can include instructions for generating a standard curve. The kit may further comprise reference standards for quantifying a CTS1 antigen and/or anti-CTS1 antibodies.

Kits may also include quality control components (e.g., sensitivity panels, calibrators, and positive controls). Preparation of quality control reagents is well-known in the art and is described on insert sheets for a variety of immunodiagnostic products. Sensitivity panel members can be used to establish assay performance characteristics, and further can be useful indicators of the integrity of the immunoassay kit reagents, and the standardization of assays.

The kit can also include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kits are provided in suitable containers as necessary, e.g., a microtiter plate. The kit can further include containers for holding or storing a sample (e.g., a container or cartridge for a urine, whole blood, plasma, or serum sample). Where appropriate, the kit can also contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

The methods and kits as described herein may also involve single molecule counting. In certain embodiments, a method for analyte analysis may involve assessing an analyte present in a sample. In certain embodiments, the assessing may be used for determining presence of and/or concentration of an analyte in a sample. In certain embodiments, the method may also be used for determining presence of and/or concentration of a plurality of different analytes present in a sample. Any device known in the art that allows for the detection of a single molecule of one or more analytes of interest can be used in the systems described herein. For example, the device can be a microfluidics device, digital microfluidics device (DMF), a surface acoustic wave based microfluidic device (SAW), an integrated DMF and analyte detection device, an integrated SAW and analyte detection device, or robotics based assay processing unit. Examples of other devices that can be used include the Quanterix SIMOA™ (Lexington, MA), Singulex's single molecule counting (SMC™) technology (Alameda, CA, see for example, U.S. Pat. No. 9,239,284, the contents of which are herein incorporated by reference), etc. Other methods of detection include the use of or can be adapted for use on a nanopore device or nanowell device. Examples of nanopore devices are described in International Patent Publication No. WO 2016/161402, which is hereby incorporated by reference in its entirety. Examples of nanowell device are described in International Patent Publication No. WO 2016/161400, which is hereby incorporated by reference in its entirety.

### 3. THERAPEUTIC METHODS

Embodiments of the present disclosure also include a method for assessing a sample from a subject for a coccidioidomycosis infection. In accordance with these embodiments, the method includes performing a reaction to detect the CTS1 detection agent bound to the recombinant CTS1 polypeptide in any of the compositions described herein, wherein CTS1 present in the sample inhibits binding of the CTS1 detection agent to the recombinant CTS1 polypeptide, thereby indicating the presence of CTS1 in the sample.

Embodiments of the present disclosure also include a method for assessing a sample from a subject for a coccidioidomycosis infection. In accordance with these embodiments, the method includes performing a reaction to detect the CTS1 detection agent bound to the recombinant CTS1 polypeptide in any of the compositions described herein, wherein anti-CTS1 antibodies present in the sample inhibit binding of the CTS1 detection agent to the recombinant CTS1 polypeptide, thereby indicating the presence of the anti-CTS1 antibodies in the sample.

Embodiments of the present disclosure also include a method for assessing a sample from a subject for a coccidioidomycosis infection. In accordance with these embodiments, the method includes performing a reaction to detect the CTS1 detection agent bound to the recombinant CTS1 polypeptide in any of the compositions described herein, wherein CTS1 present in the sample inhibits binding of the CTS1 detection agent to the recombinant CTS1 polypeptide, thereby indicating the presence of CTS1 in the sample.

Embodiments of the present disclosure also include a method for assessing a sample from a subject for a coccidioidomycosis infection, the method comprising: performing a reaction to detect the CTS1 detection agent bound to the recombinant CTS1 polypeptide in any of the compositions described herein, wherein anti-CTS1 antibodies present in the sample inhibit binding of the CTS1 detection agent to the recombinant CTS1 polypeptide, thereby indicating the presence of the anti-CTS1 antibodies in the sample.

In some embodiments, therapeutic compositions and/or methods can be used to treat and/or prevent an infection caused by Coccidioides spp, as would be recognized by one of ordinary skill in the art based on the present disclosure. In some embodiments, the treating and/or the preventing the infection comprises treating one or more symptoms of the infection in order to lessen or alleviate these symptoms.

In accordance with these embodiments, treatment may include administration of a therapeutic composition to treat the infection. In some embodiments, the therapeutic composition is administered in a single dose, and wherein the single dose treats and/or prevents at least one symptom associated with the ocular disease. In other embodiments, the composition is administered as part of a multi-dose regimen (i.e., more than one dose), and the dosing regimen treats and/or prevents at least one symptom associated with the ocular disease. In some embodiments, a multi-dose regimen includes administering a first dose, followed by at least a second dose. In some embodiments, for example, the second dose is administered about one month after the first dose, about three months after the first dose, about six months after the first dose, about one year after the first dose, about two years after the first dose, about three years after the first dose, about four years after the first dose, about five years after the first dose, about six years after the first dose, about seven years after the first dose, about eight years after the first dose, about nine years after the first dose, about ten years after the first dose. In some embodiments, the second dose is administered more than ten years after the first dose. In accordance with these embodiments, the multi-dose regimen can include a third, fourth, fifth, sixth, seventh, eighth, ninth, or tenth dose.

### 4. MATERIALS AND METHODS

Production and Purification of Recombinant CTS1. The CTS1 gene was provided to us as a kind gift from Dr Mitch Magee (Arizona State University). CTS1 was cloned into pcDNA3.1 V5/HisA, verified its identity by sequence analysis and transfected it into 293F cells (Thermo Freestyle Expression System). Seven-day supernatants were harvested and recombinant CTS1 (rCTS1) was purified on a nickel column via a C-terminal histidine tag. Purified protein was quantified using a BCA protein assay kit (Thermo Scientific) and frozen at −80° C. in 250-μL aliquots at 1 mg/mL.

Monoclonal Antibody Generation and Purification. Mice were immunized and boosted with rCTS1 mixed with Magic Mouse Adjuvant (Creative Diagnostics) under an Institutional Animal Care and Use Committee-approved protocol at Mayo Clinic. Anti-CTS1 antibody titers were monitored by rCTS1-coated 96-well plates. When a sufficient antibody titer was reached (>1:32 000), mice were killed and spleens were processed into single-cell suspensions. Splenocytes were fused with myeloma cells (P3X63Ag8.653) by a standard hybridoma generation technique. In brief, splenocytes were fused with P3X63Ag8.653 myeloma cells at a ratio of 1 splenocyte: 2 myeloma cells using 50% polyethylene glycol solution (Sigma-Aldrich). The fused cells were resuspended in hypoxanthineaminopterin-thymidine selective medium (Sigma-Aldrich) and plated at 50 000 splenocytes per well in a 96-well plate. Plates were incubated at 37° C. with 5% CO2 for 10 days. Supernatant was collected and tested by indirect ELISA for antibodies against rCTS1. Positive wells were subcloned by limiting dilution of 1 cell per well and rescreened using the same procedure after 10 days. Positive subclones were cultured in 10% fetal bovine serum complete Dulbecco's modified Eagle medium for antibody purification by protein A/G (Thermo Scientific) chromatography. Multiple monoclonal antibodies (mAbs) against rCTS1 were identified, but one in particular, 2F11, performed well in the inhibition assay.

Western Blot. rCTS1 was either treated with PNGaseF or untreated and subjected to sodium dodecyl-sulfate polyacrylamide gel electrophoresis under reducing conditions using 12% polyacrylamide gels at 140 V for 60 minutes (Bio-Rad) followed by staining with Coomassie Blue dye. For Western blot analysis, electrophoresed proteins were transferred to a polyvinylidene difluoride membrane using a Western blot apparatus (Bio-Rad). After transfer, membranes were blocked in 1% bovine serum albumin (BSA) for at least 1 hour followed by incubation with anti-CTS1 mouse monoclonal antibody 2F11 at 2 μg/mL in 1% BSA in phosphate buffered saline (PBS) for 1 hour. Membranes were then washed 3 times with Tris-buffered saline, 0.1% Tween-20 (TBST) followed by addition of peroxidase-conjugated goat anti-mouse IgG antibody (Jackson ImmunoResearch) at a dilution of 1:5000 for 45 minutes. Membranes were subsequently washed 4 times with TBST followed by addition of nitro blue tetrazolium/5-bromo-4-chloro-3-indolyl-phosphate (Thermo Scientific) substrate for development.

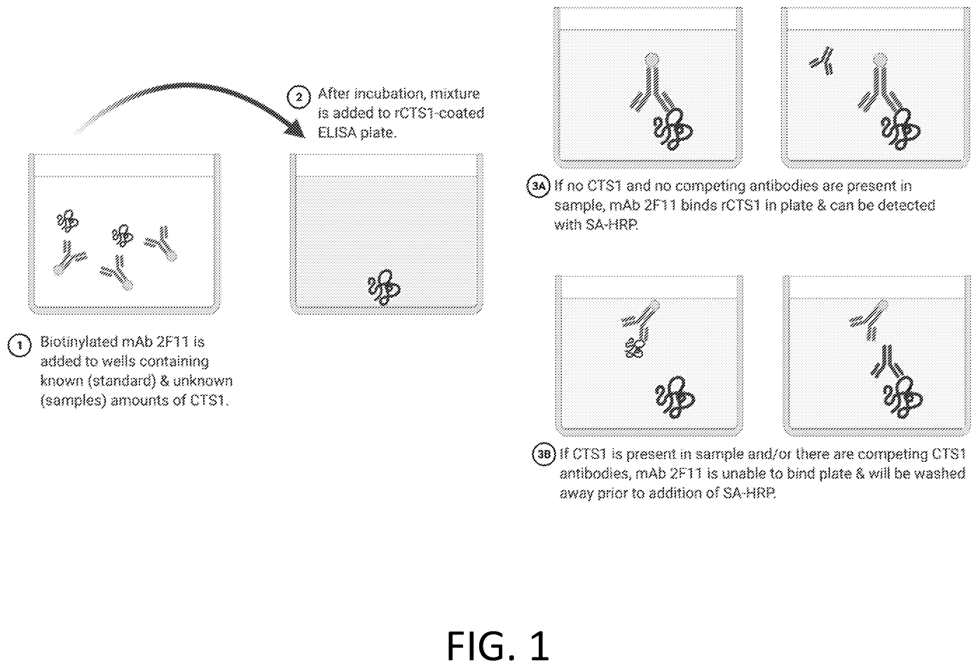

The first step of the inhibition assay requires preincubating a biofluid potentially containing Coccidioides-produced CTS1 with a calibrated concentration of biotinylated 2F11 anti-CTS1 mAb. Then, the solution is transferred to rCTS1-coated ELISA plates (FIG. 1). A sample that does not contain any CTS1 antigen or anti-CTS1 antibodies would result in 2F11 mAb binding to rCTS1 coated on the plate, whereas a sample containing CTS1 antigen or anti-CTS1 antibodies that overlap binding of 2F11 would inhibit 2F11 mAb from binding to plate-bound rCTS1. A standard of rCTS1 was run with each test so that CTS1 in the biofluid could be compared at an appropriate dilution along the linear portion of the standard curve. Biofluids that may contain anti-CTS1 antibodies that overlap with the 2F11 mAb were reported in EIA units in place of utilizing the standard curve. Commercial antigen preparations and human sera were tested undiluted and at 2-fold dilutions in 1% BSA. The assay was performed using a 96-well flat-bottom plate (Coming) coated with rCTS1 at 2 μg/mL for 75 minutes at 37° C. followed by blocking overnight in 1% BSA in 1×PBS at 4° C. The following day, rCTST standard was spiked in a 96-well U-bottom plate (Coming) at a known concentration (16 μg/mL) into 1% BSA or normal donor serum followed by ten 2-fold dilutions into 1% BSA. Biotinylated 2F11 mAb was added to either commercial antigen preparations or sera at an equal volume such that the final concentration of 2F11 was 0.1 μg/mL, final dilution of standard 8 μg/mL, and final dilution of samples 2-fold greater than starting dilution (e.g., starting dilution of 1:2 became 1:4). Samples were mixed by gently tapping the 96-well U-bottom plates followed by incubation at room temperature for 1 hour. Samples were then transferred to rCTS1-coated ELISA plates and incubated an additional hour. ELISA plates were then washed 3 times with 1×PBS, 0.05% Tween-20 (PBST), followed by addition of streptavidin-horseradish peroxidase (BD Biosciences) at a dilution of 1:5000 and incubated for 45 minutes. The plates were washed 3 times in PBST, then developed with 3,3′,5,5′-Tetramethylbenzidine substrate (BD Biosciences) for 10 minutes. Sulfuric acid 0.16 M was added to stop development and the plate was read at 450 nm.

Assay Limits and Precision. A standard curve for quantification of CTS1 was generated by spiking a known concentration of rCTST into either 1% BSA or normal donor serum followed by 2-fold dilutions into 1% BSA. The limit of blank (LOB) and limit of detection (LOD) were determined by measuring the standard curve in triplicate. The LOB was calculated by taking the mean optical density (OD) value of triplicate blank samples and subtracting 1.645 times their standard deviation (SD). Subtraction instead of addition of SD was used due to the reverse nature of the standard curve (low OD corresponds to high concentration of CTS1 whereas high OD corresponds to low concentration of CTS1). The LOD was determined by using the LOB and replicates of the CTS1 standard with concentrations that approached the LOB with the following equation: LOD=LOB−1.645(SDlow concentration sample). Once again, subtraction of SD was utilized in place of addition due to the reverse nature of the standard curve. The OD value of LOD was then backcalculated to a concentration of CTS1 to account for minor day-to-day variation. This process was repeated across 3 days for both rCTS1 spiked into 1% BSA and normal donor serum, with dilutions into 1% BSA. The average LOD concentration is reported. The back-calculated concentrations from these replicates were used to calculate intra- and interassay precision of the CTS1 standard (Table 2). For human serum tested, in place of back-calculation, OD₄₅₀ values were normalized by dividing the patient OD₄₅₀ value by the average of the negative control per plate. Normalized values were multiplied by a factor of 100 to convert data into more comprehensible integers, termed EIA units. Dilution was accounted for by dividing by n, where n is the exponent of dilution in 2”. Division instead of multiplication of dilution factor is utilized due to the reverse nature of the assay. The numbers reported are EIA units, which are arbitrary to the assay and do not discriminate if antigen or antibody is being detected.

TABLE 2
Intra- and inter-assay variation of the Coccidioidal CTS1 quantification
ELISA. Standard curves were run in triplicate on six plates over
multiple days. Intra-assay coefficient of variation (CV) was calculated
for back-calculated concentrations within each plate and the average
intra-assay CV across all plates for each standard is the value
shown. Inter-assay CV was calculated using the mean back-calculated
concentration for each standard across all plates (mean of triplicate
means). CV % is calculated by dividing the standard deviation by
the mean and converting to a percentage (×100).

| Parameter | Standard (ug/ml) | CV (%) |
|---|---|---|
| Intra-assay | 8.000 | 10.31 |
| | 4.000 | 6.37 |
| | 2.000 | 7.42 |
| | 1.000 | 8.66 |
| | 0.500 | 6.91 |
| | 0.250 | 10.65 |
| Inter-assay | 8.000 | 10.39 |
| | 4.000 | 8.85 |
| | 2.000 | 9.91 |
| | 1.000 | 9.59 |
| | 0.500 | 8.04 |
| | 0.250 | 9.21 |

Clinical Specimens. Human sera were obtained under Arizona State University Institutional Review Board (IRB) 0601000548 and Mayo Clinic IRB 12-000965. Samples were randomly selected from those sent for at least 1 routine Coccidioides-related diagnostic test and were collected between May and September 2018 and stored at <−20° C. until use. Samples were tested in dilution replicates of 1:2-1:32. If dilution replicate did not allow for quantification within the assay limits, samples were retested at higher dilutions. The status of each patient at time of sample collection was reviewed and categorized as “proven,” “probable,” or “not coccidioidomycosis” using European Organization for Research and Treatment of Cancer (EORTC) and Mycoses Study Group (MSG) criteria for endemic mycoses, with clarifying criteria for “probable” based on a published clinical composite reference standard. Any patient that was not classified as “proven,” “probable,” or “not coccidioidomycosis” was categorized as “possible.” In brief, patients who had Coccidioides identified by culture, histopathology, or PCR were classified as “proven.” Patients with concurrent clinical findings (including either radiology findings or symptoms) along with positive serology (antibodies) for Coccidioides were classified as “probable.” Anyone with either relevant clinical findings or positive serology, but not both, were classified as “possible” coccidioidomycosis; however, there is not a clear consensus about what criteria must be met for this classification. Since the true nature of the “possible” category cannot be known, these patients were excluded from the sensitivity and specificity analysis. Any patient who did not have “proven,” “probable,” or “possible” coccidioidomycosis, or was diagnosed with a different illness, was classified as “not coccidioidomycosis.”

Methods. Production and purification of recombinant CTS1. The CTS1 gene was provided from Dr. Mitch Magee (Arizona State University). CTS1 was cloned into pcDNA3.1 V5/HisA, verified its identity by sequence analysis and transfected it into 293F cells (Thermo Freestyle Expression System). Seven-day supernatants were harvested and recombinant CTS1 (rCTS1) was purified on a nickel column via a C-terminal histidine tag. Purified protein was quantified using a BCA protein assay kit (Thermo Scientific) and frozen at −80° C. in 250ul aliquots at 1 mg/ml.

Monoclonal antibody generation and purification. Mice were immunized and boosted with rCTS1 mixed with Magic Mouse Adjuvant (Creative Diagnostics) under an IACUC approved protocol at Mayo Clinic. Anti-CTS1 antibody titers were monitored by rCTS1-coated 96-well plates. When a sufficient antibody titer was reached (>1:32,000), mice were sacrificed and spleens were processed into single cell suspensions. Splenocytes were fused with myeloma cells (P3X63Ag8.653) by a standard hybridoma generation technique. Briefly, splenocytes were fused with P3X63Ag8.653 myeloma cells at a ratio of 1 splenocyte: 2 myeloma cells using 50% polyethylene glycol solution (Sigma-Aldrich). The fused cells were resuspended in hypoxanthine-aminopterin-thymidine (HAT) selective medium (Sigma-Aldrich) and plated at 50,000 splenocytes per well in a 96-well plate. Plates were incubated at 37° C. with 5% CO2 for ten days. Supernatant was collected and tested by indirect ELISA for antibodies against rCTS1. Positive wells were subcloned by limiting dilution of one cell per well and re-screened using the same procedure after ten days. Positive subclones were cultured in 10% FBS cDMEM for antibody purification by protein A/G (Thermo Scientific) chromatography. Multiple mAbs against rCTS1 were identified, but one in particular, 2F11, performed well in the inhibition assay.

Western blot. rCTS1 was either treated with PNGaseF or untreated and subjected to SDS-PAGE under reducing conditions using 12% polyacrylamide gels at 140 V for 60 minutes (Bio-Rad) followed by staining with Coomassie Blue dye. For Western blot analysis, electrophoresed proteins were transferred to a PVDF membrane using a western blot apparatus (Bio-Rad). After transfer, membranes were blocked in 1% BSA for at least one hour followed by incubation with anti-CTS1 mouse monoclonal antibody 2F11 at 2 μg/ml in 1% BSA in PBS for 1 hour. Membranes were then washed three times with tris-buffered saline, 0.10% Tween-20 (TBST) followed by addition of peroxidase-conjugated goat anti-mouse IgG antibody (Jackson ImmunoResearch) at a dilution of 1:5000 for 45 minutes. Membranes were subsequently washed four times with TBST followed by addition of nitro blue tetrazolium/5-bromo-4-chloro-3-indolyl-phosphate (Thermo Scientific) substrate for development.

Inhibition ELISA. The first step of the inhibition assay requires pre-incubating a biofluid potentially containing Coccidioides-produced CTS1 with a calibrated concentration of biotinylated 2F111 anti-CTS1 mAb. Then, the solution is transferred to rCTS1-coated ELISA plates (FIG. 1). A sample that does not contain any CTS1 antigen or anti-CTS1 antibodies would result in 2F11 mAb binding to rCTS1 coated on the plate, whereas a sample containing CTS1 antigen or anti-CTS1 antibodies that overlap binding of 2F11 would inhibit 2F11 mAb from binding to plate-bound rCTS1. A standard of rCTS1 was run with each test so that CTS1 in the biofluid could be compared at an appropriate dilution along the linear portion of the standard curve. Results for biofluids that may contain anti-CTS1 antibodies were reported in EIA units in place of utilizing the standard curve, which quantifies detection of antigen alone.

Commercial antigen preparations and human sera were tested undiluted and at 2-fold dilutions in 1% BSA. The assay was performed using a 96-well flat bottom plate (Coming) coated with rCTS1 at 2ug/ml for 75 minutes at 37° C. followed by blocking overnight in 1% BSA in 1×PBS at 4° C. The following day, rCTS1 standard was spiked in a 96-well U-bottom plate (Coming) at a known concentration (16 ug/ml) into 1% BSA or normal donor serum followed by ten 2-fold dilutions into 1% BSA. Biotinylated 2F111 mAb was added to either commercial antigen preparations or sera at an equal volume such that the final concentration of 2F11 was 0.1 ug/ml, final dilution of standard 8ug/ml, and final dilution of samples 2-fold greater than starting dilution (e.g., starting dilution of 1:2 became 1:4). Samples were mixed by gently tapping the 96-well U-bottom plates followed by incubation at room temperature for one hour. Samples were then transferred to rCTS1-coated ELISA plates and incubated an additional hour. ELISA plates were then washed three times with 1×PBS, 0.05% Tween-20 (PBST), followed by addition of Streptavidin-HRP (BD Biosciences) at a dilution of 1:5000 and incubated for 45 minutes. The plates were washed three times in PBST, then developed with 3,3′,5,5′-Tetramethylbenzidine substrate (BD Biosciences) for ten minutes. Sulfuric acid 0.16M was added to stop development and the plate was read at 450 nm.

Assay limits and precision. A standard curve for quantification of CTS1 was generated by spiking a known concentration of rCTS1 into either 1% BSA or normal donor serum followed by two-fold dilutions into 1% BSA. The limit of blank (LOB) and limit of detection (LOD) were determined by measuring the standard curve in triplicate. The LOB was calculated by taking the mean OD value of triplicate blank samples and subtracting 1.645 times their standard deviation. Subtraction instead of addition of standard deviation was used due to the reverse nature of the standard curve (low optical density corresponds to high concentration of CTS1 while high optical density corresponds to low concentration of CTS1). The LOD was determined by using the LOB and replicates of the CTS1 standard with concentrations that approached the LOB with the following equation: LOD=LOB−1.645(SD_{low concentration sample}). Once again, subtraction of SD was utilized in place of addition due to the reverse nature of the standard curve. The LOD optical density value was then back calculated to a concentration of CTS1 to account for minor day-to-day variation. This process was repeated across three days for both rCTS1 spiked into 1% BSA and normal donor serum, with dilutions into 1% BSA. The average LOD concentration is reported. The back-calculated concentrations from these replicates were used to calculate intra- and inter-assay precision of the CTS1 standard (Table 2). For human serum tested, in place of back-calculation, OD₄₅₀ values were normalized by dividing the patient OD₄₅₀ value by the average of the negative control per plate. Normalized values were multiplied by a factor of 100 to convert data into more comprehensible integers, termed EIA units. Dilution was accounted for by dividing by n, where n is the exponent of dilution in 2”. Division instead of multiplication of dilution factor is utilized due to the reverse nature of the assay. The numbers reported are EIA units, which are arbitrary to the assay and do not discriminate if antigen or antibody is being detected.

Clinical specimens. Human sera were obtained under ASU IRB 0601000548 and Mayo Clinic IRB 12-000965. Samples were randomly selected from those sent for at least one routine Coccidioides-related diagnostic test and were collected between May-September 2018 and stored <−20° C. until use. Samples were tested in dilution replicates of 1:2-1:32. If dilution replicate did not allow for quantification within the assay limits, samples were re-tested at higher dilutions. The status of each patient at time of sample collection was reviewed and categorized as “proven”, “probable”, or “not coccidioidomycosis” using European Organization for Research and Treatment of Cancer (EORTC) and Mycoses Study Group (MSG) criteria for endemic mycoses, with clarifying criteria for “probable” based on the published clinical composite reference standard. Any patient that was not classified as “proven”, “probable” or “not coccidioidomycosis” was categorized as “possible”. Briefly, patients who had Coccidioides identified by culture, histopathology, or PCR were classified as “proven”. Patients with concurrent clinical findings (including either radiology findings or symptoms) along with positive serology (antibodies) for Coccidioides were classified as “probable”. Anyone with either relevant clinical findings or positive serology, but not both, were classified as “possible” coccidioidomycosis, however there is not a clear consensus about what criteria must be met for this classification. Since the true nature of the “possible” category cannot be known, these patients were excluded from the sensitivity and specificity analysis. Any patient who did not have “proven”, “probable”, or “possible” coccidioidomycosis, or was diagnosed with a different illness, was classified as “not coccidioidomycosis.”

Patient Consent Statement. This study did not include factors necessitating patient consent.

Statistical Analysis. Receiver operating characteristic analysis was used to determine the cutoff for positivity as well as estimate sensitivity and specificity.

### 5. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

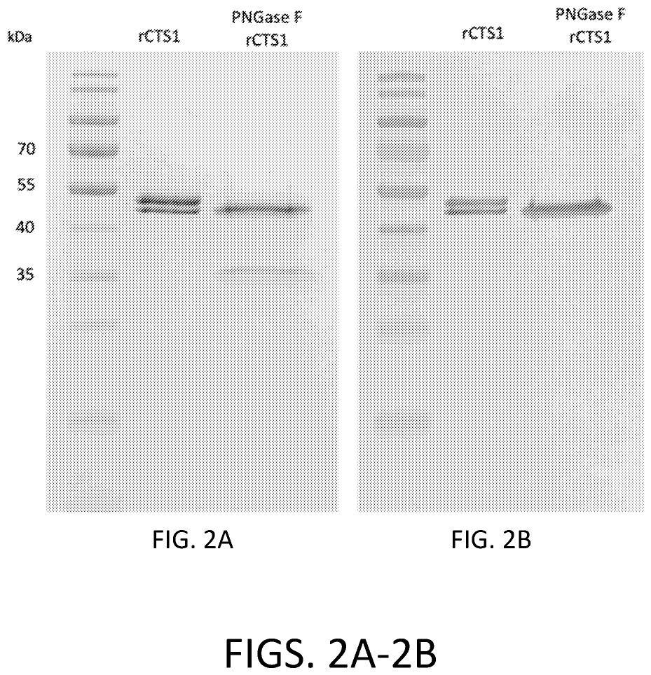

Recombinant CTS1 was electrophoresed with and without pretreatment with PNGase F (FIG. 2A). rCTS1 appears as a doublet band, which is presumed to be a glycosylation since rCTS1 appears as a single band after deglycosylation with PNGase F (˜35 kDa). Although multiple mAbs were identified from anti-CTS1-secreting mouse hybridomas, one mAb in particular, 2F111, demonstrated binding in both ELISA and Western blotting. A Western blot of 2F11 reacting with rCTS1 is shown in FIG. 2B. This antibody was used to develop the inhibition assay. Dilutions of rCTS1 were used in an empirical approach to establish the limit of blank, which was then used to calculate the limit of detection of Coccidioides rCTS1, 155 ng/mL (SD, 0.022 μg/mL).

### Example 2

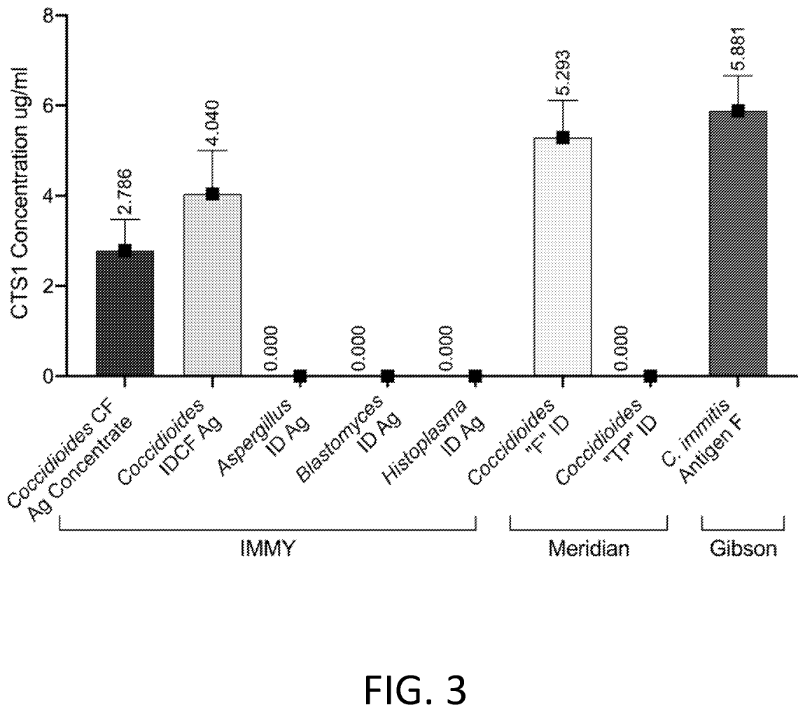

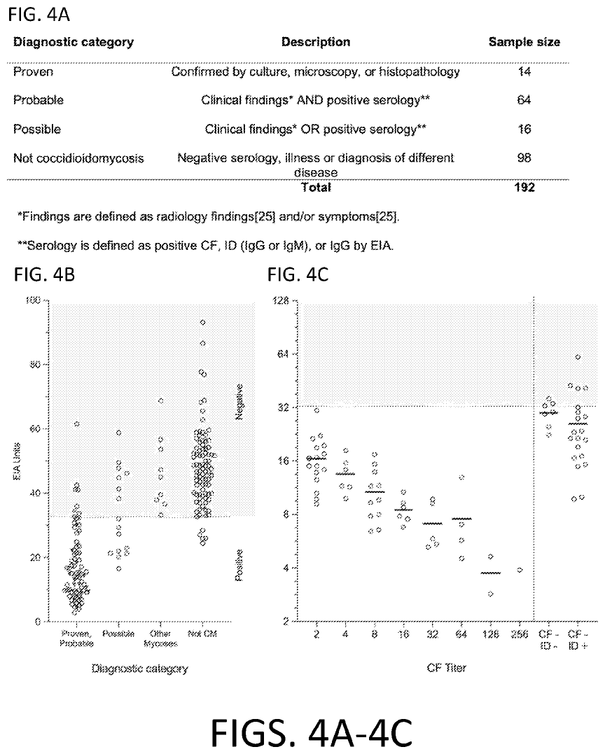

The first step of characterizing the assay was to test it against commercially available antigen preparations. These included Coccidioides CF Antigen Concentrate (IMMY, Norman, Oklahoma), Coccidioides IDCF Antigen (IMMY), Coccidioides “F” Antigen for Immunodiffusion (Meridian Biosciences, Cincinnati, Ohio), and Coccidioides immitis Antigen F (Gibson Bioscience, Lexington, Kentucky). The quantity of CTS1 in each was 2.79 μg/mL, 4.04 μg/mL, 5.29 μg/mL, and 5.88 μg/mL, respectively (FIG. 3). Commercially available antigen preparations for Aspergillus, Blastomyces, and Histoplasma ID assays (IMMY) were also tested, but no CTS1 was detected in noncoccidioidal antigen preparations, demonstrating the specificity of 2F11 mAb (FIG. 3). The same inhibition ELISA format was used to test 192 preexisting serum samples, of which 78 (40.6%) patients had proven or probable coccidioidomycosis, 16 (8.3%) had possible coccidioidomycosis, and the remaining 98 (51.0%) did not have coccidioidomycosis (FIG. 4A). Of the 98 samples classified as not coccidioidomycosis, 9 (4.7%) were diagnosed with a different fungal infection (Aspergillus n=6, by Aspergillus galactomannan antigen, and Candida n=3, by fungal culture). Mean EIA units of dilution replicates for all samples tested are shown in FIG. 4B. Of the 78 patients with proven or probable coccidioidomycosis, 51 had a positive CF titer ranging from 1:2 to 1:256. The mean EIA units of these patients in relation to CF titer, as well as in patients without a CF titer, is shown in FIG. 4C. Importantly, 4 patient serum samples that did not have CF titers and were negative for ID show reactivity in the assay (FIG. 4C).

### Example 3

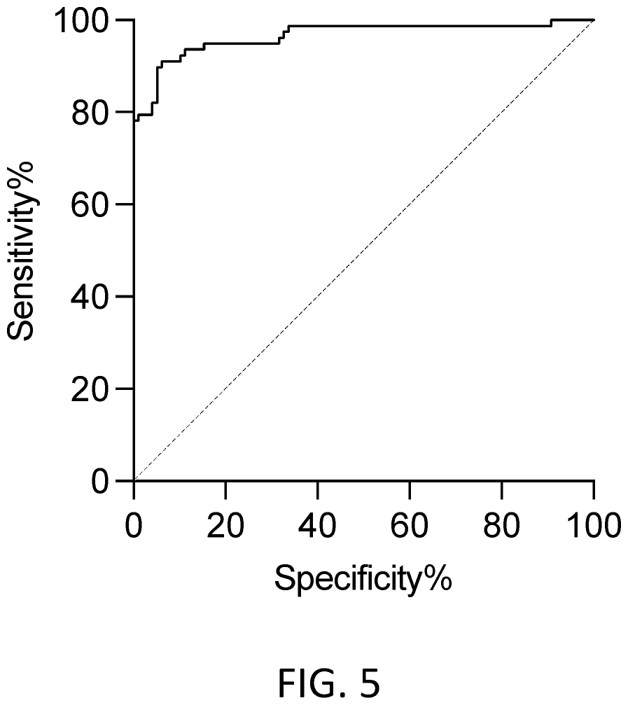

A receiver operating characteristic curve was plotted using patients with proven or probable coccidioidomycosis as true positives and patients without coccidioidomycosis as true negatives (FIG. 5) while excluding the possible group, as their status is unknown. The area under the receiver operating characteristic curve was 0.9652 (standard error, 0.01441 [95% confidence interval, 0.9370-0.9934]; P<0.0001) using a positive cutoff value of 32.5 EIA units, resulting in a sensitivity and specificity of 89.74% and 94.90%, respectively.

### Example 4

An inhibition-based enzyme-linked immunoassay (ELISA) was developed that utilizes a monoclonal antibody specific for coccidioidal CTS1. CTS1 was quantified in commercial antigen preparations using recombinant CTS1 as a standard. Sera from 192 individuals from an endemic area were tested which included 78 patients (40.6%) with proven or probable coccidioidomycosis.

The quantity of CTS1 in diagnostic commercial antigen preparations from different suppliers varied. Temporal constraints of availability of different lots of commercial antigens does not allow for immediate comparison of lot-to-lot variability. Assay results from patient serum samples correlated with low- and high-titer serology from patients with a coccidioidomycosis diagnosis. Further analysis suggested that patient derived anti-CTS1 antibodies may overlap with the mouse monoclonal antibody used in the assay. This unexpected overlap in CTS1 binding suggests the assay can detect antigen, antibody, or both, which contributes to its high level of clinical sensitivity of 89.74% and specificity of 94.90%.

Recombinant CTS1 was electrophoresed with and without pre-treatment with PNGase F (FIG. 2A). rCTS1 appears as a doublet band, which is presumed to be a glycosylation since rCTS1 appears as a single band after deglycosylation with PNGase F (˜35 kDa). Although multiple mAbs were identified from anti-CTS1-secreting mouse hybridomas, one mAb in particular, 2F11, demonstrated binding in both ELISA and western blotting. A western blot of 2F11 reacting with rCTS1 is shown in FIG. 2B. This antibody was used to develop the inhibition assay. Dilutions of rCTS1 were used in an empirical approach to establish the limit of blank, which was then used to calculate the limit of detection of Coccidioides rCTS1, 155 ng/ml (SD 0.022 μg/ml).

The first step of characterizing the assay was to test it against commercially available antigen preparations. These included Coccidioides CF Antigen Concentrate (IMMY, Norman, OK), Coccidioides IDCF Antigen (IMMY, Norman, OK), Coccidioides “F” Antigen for Immunodiffusion (Meridian Biosciences, Cincinnati, OH), and Coccidioides immitis Antigen F (Gibson Bioscience, Lexington, KY). The quantity of CTS1 in each was 2.79 ug/ml, 4.04 ug/ml, 5.29 ug/ml, and 5.88 ug/ml, respectively (FIG. 3). Commercially available antigen preparations for Aspergillus, Blastomyces, and Histoplasma ID assays (IMMY, Norman, OK) were also tested, but no CTS1 was detected in non-coccidioidal antigen preparations, demonstrating the specificity of 2F11 mAb (FIG. 3).

The same inhibition ELISA format was used to test 192 pre-existing serum samples, of which 78 (40.6%) patients had proven or probable coccidioidomycosis, 16 (8.3%) had possible coccidioidomycosis, and the remaining 98 (51.0%) did not have coccidioidomycosis (FIG. 4A). Of the 98 samples classified as not coccidioidomycosis, 9 (4.7%) were diagnosed with a different fungal infection (Aspergillus n=6, by Aspergillus galactomannan antigen, and Candida n=3, by fungal culture). Mean EIA units of dilution replicates for all samples tested are shown in FIG. 4B. Of the 78 patients with proven or probable coccidioidomycosis, 51 had a positive complement fixation titer ranging from 1:2 to 1:256. The mean EIA units of these patients in relation to CF titer, as well as in patients without a CF titer, is shown in FIG. 4C. Importantly, four patient serum samples that did not have CF titers and were negative for ID show reactivity in the assay (FIG. 4C).

A receiver operating characteristic curve (ROC) was plotted using patients with proven or probable coccidioidomycosis as true positives and not coccidioidomycosis patients as true negatives (FIG. 5) while excluding the possible group, as their status is unknown. The area under the receiver operating characteristic curve was 0.9652 (SE, 0.01441; 95% CI, 0.9370-0.9934; P<0.0001) using a positive cutoff value of 32.5 EIA units, resulting in a sensitivity and specificity of 89.74% and 94.90%, respectively.

Taken together, these results demonstrate that the CTS1 inhibition ELISA described in the present disclosure is a promising tool to aid in quality control of antigens used in the diagnosis of coccidioidomycosis.

It is understood that the foregoing detailed description and accompanying examples are merely illustrative and are not to be taken as limitations upon the scope of the embodiments of the present disclosure, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the embodiments of the present disclosure, may be made without departing from the spirit and scope thereof.

For reasons of completeness, various aspects of the embodiments of the present disclosure are set out in the following numbered clauses:

Clause 1. A composition for detecting and/or quantifying Coccidioidal Chitinase-1 (CTS1) antigen and/or anti-CTS1 antibodies, the composition comprising: a CTS1 detection agent comprising a CTS1 target antigen binding domain and a detection moiety; and a recombinant CTS1 polypeptide comprising a CTS1 target antigen detectable by the CTS1 detection agent.

Clause 2. The composition of clause 1, wherein the composition further comprises a sample from a subject, wherein: (a) CTS1 present in the sample inhibits binding of the CTS1 detection agent to the recombinant CTS1 polypeptide, thereby facilitating detection of the CTS1 in the sample; and/or (b) anti-CTS1 antibodies present in the sample inhibit binding of the CTS1 detection agent to the recombinant CTS1 polypeptide, thereby facilitating detection of the anti-CTS1 antibodies in the sample.

Clause 3. The composition of clause 1 or clause 2, wherein the CTS1 detection agent comprises a CTS1 antibody or a CTS1 target antigen binding fragment thereof.

Clause 4. The composition of any one of clauses 1 to 3, wherein the detection moiety comprises at least one of a peptide, a polypeptide, a fluorophore, a chromophore, a radiolabel, a nucleotide, a polynucleotide, a small molecule, an enzyme, and/or a nanoparticle.

Clause 5. The composition of any one of clauses 1 to 4, wherein the small molecule is biotin.

Clause 6. The composition of any one of clauses 1 to 5, wherein the polypeptide is streptavidin.

Clause 7. The composition of clause 2, wherein the sample is a serum sample from a subject that is infected with or suspected of being infected with Coccidioides spp.

Clause 8. The composition of any one of clauses 1 to 7, wherein the recombinant CTS1 polypeptide is attached to a substrate.

Clause 9. The composition of clause 8, wherein the substrate comprises glass, silicon, a metal oxide, and/or a polymer.

Clause 10. The composition of any one of clauses 1 to 9, wherein the composition further comprises a CTS1 calibrator or control polypeptide.

Clause 11. A method for assessing a sample from a subject for a coccidioidomycosis infection, the method comprising: performing a reaction to detect the CTS1 detection agent bound to the recombinant CTS1 polypeptide in the composition of any one of clauses 1 to 10, wherein CTS1 present in the sample inhibits binding of the CTS1 detection agent to the recombinant CTS1 polypeptide, thereby indicating the presence of CTS1 in the sample.

Clause 12. A method for assessing a sample from a subject for a coccidioidomycosis infection, the method comprising: performing a reaction to detect the CTS1 detection agent bound to the recombinant CTS1 polypeptide in the composition of any one of clauses 1 to 10, wherein anti-CTS1 antibodies present in the sample inhibit binding of the CTS1 detection agent to the recombinant CTS1 polypeptide, thereby indicating the presence of the anti-CTS1 antibodies in the sample.

Clause 13. A composition for detecting and/or quantifying Coccidioidal Chitinase-1 (CTS1) antigen, the composition comprising: a CTS1 detection agent comprising a CTS1 target antigen binding domain and a detection moiety; and a recombinant CTS1 polypeptide comprising a CTS1 target antigen detectable by the CTS1 detection agent.

Clause 14. The composition of clause 13, wherein the composition further comprises a sample from a subject, wherein CTS1 present in the sample inhibits binding of the CTS1 detection agent to the recombinant CTS1 polypeptide, thereby facilitating detection of the CTS1 in the sample.

Clause 15. The composition of clause 13 or clause 14, wherein the CTS1 detection agent comprises a CTS1 antibody or a CTS1 target antigen binding fragment thereof.

Clause 16. The composition of any one of clauses 13 to 15, wherein the detection moiety comprises at least one of a peptide, a polypeptide, a fluorophore, a chromophore, a radiolabel, a nucleotide, a polynucleotide, a small molecule, an enzyme, and/or a nanoparticle.

Clause 17. The composition of clause 14, wherein the sample is a serum sample from a subject that is infected with or suspected of being infected with Coccidioides spp.

Clause 18. The composition of any one of clauses 13 to 17, wherein the recombinant CTS1 polypeptide is attached to a substrate.

Clause 19. The composition of any one of clauses 13 to 18, wherein the composition further comprises a CTS1 calibrator or control polypeptide.

Clause 20. A method for assessing a sample from a subject for a coccidioidomycosis infection, the method comprising: performing a reaction to detect the CTS1 detection agent bound to the recombinant CTS1 polypeptide in the composition of any clauses 13 to 19, wherein CTS1 present in the sample inhibits binding of the CTS1 detection agent to the recombinant CTS1 polypeptide, thereby indicating the presence of CTS1 in the sample.

Clause 21. A composition for detecting and/or quantifying anti-Coccidioidal Chitinase-1 (CTS1) antibodies, the composition comprising: a CTS1 detection agent comprising a CTS1 target antigen binding domain and a detection moiety; and a recombinant CTS1 polypeptide comprising a CTS1 target antigen detectable by the CTS1 detection agent.

Clause 22. The composition of clause 21, wherein the composition further comprises a sample from a subject, wherein anti-CTS1 antibodies present in the sample inhibit binding of the CTS1 detection agent to the recombinant CTS1 polypeptide, thereby facilitating detection of the anti-CTS1 antibodies in the sample.

Clause 23. The composition of clause 21 or clause 22, wherein the CTS1 detection agent comprises a CTS1 antibody or a CTS1 target antigen binding fragment thereof.

Clause 24. The composition of any one of clauses 21 to 23, wherein the detection moiety comprises at least one of a peptide, a polypeptide, a fluorophore, a chromophore, a radiolabel, a nucleotide, a polynucleotide, a small molecule, an enzyme, and/or a nanoparticle.

Clause 25. The composition of clause 22, wherein the sample is a serum sample from a subject that is infected with or suspected of being infected with Coccidioides spp.

Clause 26. The composition of any one of clauses 21 to 25, wherein the recombinant CTS1 polypeptide is attached to a substrate.

Clause 27. The composition of any one of clauses 21 to 26, wherein the composition further comprises a CTS1 calibrator or control polypeptide.

Clause 28. A method for assessing a sample from a subject for a coccidioidomycosis infection, the method comprising: performing a reaction to detect the CTS1 detection agent bound to the recombinant CTS1 polypeptide in the composition of any one of clauses 21 to 27, wherein anti-CTS1 antibodies present in the sample inhibit binding of the CTS1 detection agent to the recombinant CTS1 polypeptide, thereby indicating the presence of the anti-CTS1 antibodies in the sample.

Clause 29. A kit for detecting and/or quantifying Coccidioidal Chitinase-1 (CTS1) antigen and/or anti-CTS1 antibodies, the kit comprising: a CTS1 detection agent comprising a CTS1 target antigen binding domain and a detection moiety; and a recombinant CTS1 polypeptide comprising a CTS1 target antigen detectable by the CTS1 detection agent.

Clause 30. The kit of clause 29, wherein the kit comprises a CTS1 calibrator or control polypeptide.

Clause 31. The kit of clause 29 or clause 30, wherein the kit comprises instructions for performing an assay to detect and/or quantify Coccidioidal Chitinase-1 (CTS1) antigen and/or anti-CTS1 antibodies in a sample from a subject.

### 6. SEQUENCES

The various embodiments described herein include the use of (or make reference to) following nucleotide and amino acid sequences.

2F11-B9 VH3 (SEQ ID NO: 1):
CAGGTTCAGCTGGAGGAGTCTGGGGGAGGCTTAGTGAAGCCTGGAGGG
TCCCTGAAACTCTCCTGTGCAGCCCCTGGATTCACTTTCAGTAGCTATG
ACATGTCTTGGGTTCGCCAGACTCCGGAGAAGAGGCTGGAGTGGGTCGC
AACCATTAGTGGTGGTGGTAGTTACACCTACTATCCAGACAGTGTGAAG
GGGCGATTCACCATCTCCAGAGACAATGCCAAGAACAACCTGTATCTGC
AAATGAGCAGTCTGAGGTCTGAGGACACGGCCTTGTATTACTGTGCAAG
ACAGGGTAGTAGCCACTGGTACTTCGATGTCTGGGGCGCAGGGACCACG
GTCACCGTCTCCTCA
2F11-B9 VLK (SEQ ID NO: 2):
GATATTGTGATGACACAGACTCCAGCAATCATGTCTGCATCTCCAGGGG
AGAAGGTCACCATGACCTGCAGTGCCAGCTCAAGTGTAAGTTACATGCA
CTGGTATCAGCAGAAGTCAGGCACCTCCCCCAAAAGATGGATTTATGAC
ACATCCAAACTGGCTTCTGGAGTCCCTGCTCGCTTCAGTGGCAGTGGGT
CTGGGACCTCTTACTCTCTCACAATCAGCAGCATGGAGTCTGAAGATGC
TGCCACTTATTACTGCCAGCCGTGGACTAGTAACCCACCGTACACGTTC
GGAGGGGGGACCAAGCTGGAAATAAAA
4H2-B1 VH1 (SEQ ID NO: 3):
CAGGTACAGCTGCAGGAGTCAGGGGCTGAGCTGGTGAGGCCTGGGTCC
TCAGTGCAGATTTCCTGCAAGGCTTCTGGCTATGCATTCAGTGGCTTCT
GGATGAACTGGGTGAAGCAGAGGCCTGGACAGGGTCTTGAGTGGATTGG
GCAGATTTATCCTGGAGATGGTGACACTAAATACAATGGAAACTTCAAG
GATAAAGGCACACTGACTGTAGACAAGTCCTCCAGCACAGTCTACATGC
AGCTCAGCAGCCTAACATCTGAGGACTCTGCGGTCTATTTCTGTGCAAG
AGGAAGGAGTCAGCTCGGGCCCTTTGCTTACTGGGGCCAAGGGACTCTG
GTCACTGTCTCT
4H2-B1 VLK (SEQ ID NO: 4):
GATATTGTGATGACACAATCTCCAGCATTCATGTCTGCTTCTCCAGGGG
AGAAGGTCACCATGACCTGCACTGCCAGCTCAAGTGTAAATTACATGTA
CTGGTACCAGCAGAAGCCAGGATCCTCCCCCAAACCCTGGATTTATCTC
ACATCCAACCTGGCTTCTGGAGTCCCTGCTCGCTTCAATGGCAGTGGGT
CTGGGACCTCTTACTCTCTCACAATCAGCACCATGGAGGCTGAAGATGC
TGCCACTTATTACTGCCAGCAGTGGACTAGTAAGCCACCCACGTTCGGC
TCGGGGACAAAGTTGGAAATAAAA
10F12-E7 VH1 (SEQ ID NO: 5):
CAGGTCAAGCTGGAGGAGTCTGGACCTGGCCTGGTGAAACCTTCTCAG
TCTCTGTCCCTCACCTGCACTGTCACTGGCTACTCAATCACCAGTGATT
ATGCCTGGAACTGGATCCGGCAGTTTCCAGGAAACAGGCTGGAGTGGAT
GGGCTACATAGACTACAGTGATAGCACTACCTACAACCCATCTCTCAAA
AGTCGATTCTCTCTCACTCGAGACACATCCAAGAACCAGTTCTTCCTGC
AGTTGAATTCTGTGACTACTGAGGACACAGCCACATATTACTGTGCTAG
GGGGTCTTACTATGGTAACTGGGGCTATGCTATGGACTACTGGGGTCAA
GGAACCTCAGTCACCGTCTCcTCA
10F12-E7 VLK (SEQ ID NO: 6):
GACATTGTGATCACCCAATCTCCATCCTCCCTAGCTGTGTCGGTTGGAG
AGAAGGTTACTATGAGCTGCAAGTCCAGTCAGAGCCTTTTATATAGTTA
CAATCAAAAGAACTACCTGGCCTGGTACCAGCAGAAACCAGGGCAGTCT
CCTAAATTGCTGATTCACTGGGCATCCACTAGGGCATCTGGGGTCCCTG
ATCGCTTCACAGGCAGTGGATCTGGGACAGATTTCACTCTCACCATCAG
CAGTGTGAAGGCTGAAGACCTGGCAGTTTATTACTGTCAACAATATTAT
ACCTATCCGCTCACGTTCGGTGCTGGGACCAAGCTGGAGCTGAAA
11A10-F6 VH2A (SEQ ID NO: 7):
GAGGTGCAGCTGGAGGAGTCTGGCCCTGGGATATTGCAGCCCTCCCAG
ACCCTCAGTCTGACTTGTTCTTTCTCTGGGTTTTCACTGAACACTTTTG
GTATAGGAGTAGGCTGGATTCGTCAGCCTTCAGGGAAGGGTCTGGACTG
GCTGGCACACATTTGGTGGAATGATAATAAGTCCTATAACACAGCCCTG
AAGAGCCGGCTCACAATCTCCAAGGATACCTCCAACAACCAGGTATTCC
TCAAGATCGCCAGTGTGGACACTGCAGATACTGCCACATACTACTGTGC
TCGAATAGGGGACTATGGTAACTACCCTTTTGCTTACTGGGGCCAAGGG
ACTCTGGTCACTGTCTCTGCA
11A10-F6 VLK (SEQ ID NO: 8):
GACATTGTGATGACACAGTCTACATCCTCCCTGTCTGCCTCTCTGGGAG
ACAGAGTCACCATCAGTTGCAGGGCAAGTCAGGACATTAGCAATTATTT
AACCTGGTATCAGCAGAAGCCAGATGGAGCTGTTAAACTCCTGATCTAC
TACACATCAAGATTACACTCAGGAGTCCCATCAAGGTTCAGTGGCAGTG
GGTCTGGAACAGATTATTCTCTCACCATTAGCAACCTGGAGCAAGAAGA
TATTGCCACTTACTTTTGCCAACAGGGTAATACGCTTCCGTGGACGTTC
GGTGGAGGCACCAAGCTTGAAATCAAA
16F6-H2 VH1 (SEQ ID NO: 9):
GAAGTACAGCTGGAGGAGTCTGGGGGAGGCTTAGTGAAGCCTGGAGGG
TCCCTGACACTCTCCTGTGCAGCCTCTGGATTCACTTTCAGTAACTATG
GCATGTCTTGGGTTCGCCAGTCTCCAGAGAAGAGGCTGGAGTGGGTCGC
AGAAATCAGTAGTGGTAGTAGCTATATTTACTATCCAGACACTGTGACG
GGCCGATTCACCATCTCCAGAGACAATGCCAAGAACACCCTGTACCTGG
AAATGAACAGTCTGAGGTCTGACGACACGGCCATGTATTACTGTGCAAG
GGGTCCTAGTATGATTTCGCCGTTCTACTTTGACTACTGGGGCCAAGGC
ACCACTCTCACAGTCTCC
16F6-H2 VLK (SEQ ID NO: 10):
GATATTGTGCTCACCCAGTcTCCAGCATCCCTGTCCGTGGcTACAGGAG
AAAAAGTCACTATCAGATGCATAACCAGCACTGATATTGATGATGATAT
GAACTGGTACCAGCAGAAGCCAGGGGAACCTCcTAAGCTCCTTATTTCA
GAAGGCAATACTCTTCGTCCTGGAGTCCCATCCCGATTCTCCAGCAGTG
GCTATGGCACAGATTTTGTTTTTACAATTGAAAACACGCTCTCAGAAGA
TGTTGCAGATTACTACTGTTTGCAAAGTGATAACATGCCGTACACGTTC
GGAGGGGGGACCAAGCTGGAAATAAAA

1-12. (canceled)

13. A composition for detecting and/or quantifying Coccidioidal Chitinase-1 (CTS1) antigen, the composition comprising:
a CTS1 detection agent comprising a CTS1 target antigen binding domain and a detection moiety; and
a recombinant CTS1 polypeptide comprising a CTS1 target antigen detectable by the CTS1 detection agent.

14. The composition of claim 13, wherein the composition further comprises a sample from a subject, wherein CTS1 present in the sample inhibits binding of the CTS1 detection agent to the recombinant CTS1 polypeptide, thereby facilitating detection of the CTS1 in the sample.

15. The composition of claim 13, wherein the CTS1 detection agent comprises a CTS1 antibody or a CTS1 target antigen binding fragment thereof.

16. The composition of claim 13, wherein the detection moiety comprises at least one of a peptide, a polypeptide, a fluorophore, a chromophore, a radiolabel, a nucleotide, a polynucleotide, a small molecule, an enzyme, and/or a nanoparticle.

17. The composition of claim 14, wherein the sample is a serum sample from a subject that is infected with or suspected of being infected with Coccidioides spp.

18. The composition of claim 13, wherein the recombinant CTS1 polypeptide is attached to a substrate.

19. The composition of claim 13, wherein the composition further comprises a CTS1 calibrator or control polypeptide.

20. A method for assessing a sample from a subject for a coccidioidomycosis infection, the method comprising: performing a reaction to detect the CTS1 detection agent bound to the recombinant CTS1 polypeptide in the composition of claim 13, wherein CTS1 present in the sample inhibits binding of the CTS1 detection agent to the recombinant CTS1 polypeptide, thereby indicating the presence of CTS1 in the sample.

21. A composition for detecting and/or quantifying anti-Coccidioidal Chitinase-1 (CTS1) antibodies, the composition comprising:
a CTS1 detection agent comprising a CTS1 target antigen binding domain and a detection moiety; and
a recombinant CTS1 polypeptide comprising a CTS1 target antigen detectable by the CTS1 detection agent.

22. The composition of claim 21, wherein the composition further comprises a sample from a subject, wherein anti-CTS1 antibodies present in the sample inhibit binding of the CTS1 detection agent to the recombinant CTS1 polypeptide, thereby facilitating detection of the anti-CTS1 antibodies in the sample.

23. The composition of claim 21, wherein the CTS1 detection agent comprises a CTS1 antibody or a CTS1 target antigen binding fragment thereof.

24. The composition of claim 21, wherein the detection moiety comprises at least one of a peptide, a polypeptide, a fluorophore, a chromophore, a radiolabel, a nucleotide, a polynucleotide, a small molecule, an enzyme, and/or a nanoparticle.

25. The composition of claim 22, wherein the sample is a serum sample from a subject that is infected with or suspected of being infected with Coccidioides spp.

26. The composition of claim 21, wherein the recombinant CTS1 polypeptide is attached to a substrate.

27. The composition of claim 21, wherein the composition further comprises a CTS1 calibrator or control polypeptide.

28. A method for assessing a sample from a subject for a coccidioidomycosis infection, the method comprising: performing a reaction to detect the CTS1 detection agent bound to the recombinant CTS1 polypeptide in the composition of claim 21, wherein anti-CTS1 antibodies present in the sample inhibit binding of the CTS1 detection agent to the recombinant CTS1 polypeptide, thereby indicating the presence of the anti-CTS1 antibodies in the sample.

29. A kit for detecting and/or quantifying Coccidioidal Chitinase-1 (CTS1) antigen and/or anti-CTS1 antibodies, the kit comprising:
a CTS1 detection agent comprising a CTS1 target antigen binding domain and a detection moiety; and
a recombinant CTS1 polypeptide comprising a CTS1 target antigen detectable by the CTS1 detection agent.

30. The kit of claim 29, wherein the kit comprises a CTS1 calibrator or control polypeptide.

31. The kit of claim 29, wherein the kit comprises instructions for performing an assay to detect and/or quantify Coccidioidal Chitinase-1 (CTS1) antigen and/or anti-CTS1 antibodies in a sample from a subject.


SEQUENCE LISTING (10 sequences)

SEQ ID NO: 1 - DNA, 357 bp, synthetic construct
  source 1..357: mol_type = other DNA
caggttcagctggaggagtctgggggaggcttagtgaagcctggagggtccctgaaactctcctgtgcagcccctggattcactttcagtagctatgacatgtcttgggttcgccagactccggagaagaggctggagtgggtcgcaaccattagtggtggtggtagttacacctactatccagacagtgtgaaggggcgattcaccatctccagagacaatgccaagaacaacctgtatctgcaaatgagcagtctgaggtctgaggacacggccttgtattactgtgcaagacagggtagtagccactggtacttcgatgtctggggcgcagggaccacggtcaccgtctcctca

SEQ ID NO: 2 - DNA, 321 bp, synthetic construct
  source 1..321: mol_type = other DNA
gatattgtgatgacacagactccagcaatcatgtctgcatctccaggggagaaggtcaccatgacctgcagtgccagctcaagtgtaagttacatgcactggtatcagcagaagtcaggcacctcccccaaaagatggatttatgacacatccaaactggcttctggagtccctgctcgcttcagtggcagtgggtctgggacctcttactctctcacaatcagcagcatggagtctgaagatgctgccacttattactgccagccgtggactagtaacccaccgtacacgttcggaggggggaccaagctggaaataaaa

SEQ ID NO: 3 - DNA, 354 bp, synthetic construct
  source 1..354: mol_type = other DNA
caggtacagctgcaggagtcaggggctgagctggtgaggcctgggtcctcagtgcagatttcctgcaaggcttctggctatgcattcagtggcttctggatgaactgggtgaagcagaggcctggacagggtcttgagtggattgggcagatttatcctggagatggtgacactaaatacaatggaaacttcaaggataaaggcacactgactgtagacaagtcctccagcacagtctacatgcagctcagcagcctaacatctgaggactctgcggtctatttctgtgcaagaggaaggagtcagctcgggccctttgcttactggggccaagggactctggtcactgtctct

SEQ ID NO: 4 - DNA, 318 bp, synthetic construct
  source 1..318: mol_type = other DNA
gatattgtgatgacacaatctccagcattcatgtctgcttctccaggggagaaggtcaccatgacctgcactgccagctcaagtgtaaattacatgtactggtaccagcagaagccaggatcctcccccaaaccctggatttatctcacatccaacctggcttctggagtccctgctcgcttcaatggcagtgggtctgggacctcttactctctcacaatcagcaccatggaggctgaagatgctgccacttattactgccagcagtggactagtaagccacccacgttcggctcggggacaaagttggaaataaaa

SEQ ID NO: 5 - DNA, 366 bp, synthetic construct
  source 1..366: mol_type = other DNA
caggtcaagctggaggagtctggacctggcctggtgaaaccttctcagtctctgtccctcacctgcactgtcactggctactcaatcaccagtgattatgcctggaactggatccggcagtttccaggaaacaggctggagtggatgggctacatagactacagtgatagcactacctacaacccatctctcaaaagtcgattctctctcactcgagacacatccaagaaccagttcttcctgcagttgaattctgtgactactgaggacacagccacatattactgtgctagggggtcttactatggtaactggggctatgctatggactactggggtcaaggaacctcagtcaccgtctcctca

SEQ ID NO: 6 - DNA, 339 bp, synthetic construct
  source 1..339: mol_type = other DNA
gacattgtgatcacccaatctccatcctccctagctgtgtcggttggagagaaggttactatgagctgcaagtccagtcagagccttttatatagttacaatcaaaagaactacctggcctggtaccagcagaaaccagggcagtctcctaaattgctgattcactgggcatccactagggcatctggggtccctgatcgcttcacaggcagtggatctgggacagatttcactctcaccatcagcagtgtgaaggctgaagacctggcagtttattactgtcaacaatattatacctatccgctcacgttcggtgctgggaccaagctggagctgaaa

SEQ ID NO: 7 - DNA, 363 bp, synthetic construct
  source 1..363: mol_type = other DNA
gaggtgcagctggaggagtctggccctgggatattgcagccctcccagaccctcagtctgacttgttctttctctgggttttcactgaacacttttggtataggagtaggctggattcgtcagccttcagggaagggtctggactggctggcacacatttggtggaatgataataagtcctataacacagccctgaagagccggctcacaatctccaaggatacctccaacaaccaggtattcctcaagatcgccagtgtggacactgcagatactgccacatactactgtgctcgaataggggactatggtaactacccttttgcttactggggccaagggactctggtcactgtctctgca

SEQ ID NO: 8 - DNA, 321 bp, synthetic construct
  source 1..321: mol_type = other DNA
gacattgtgatgacacagtctacatcctccctgtctgcctctctgggagacagagtcaccatcagttgcagggcaagtcaggacattagcaattatttaacctggtatcagcagaagccagatggagctgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttccgtggacgttcggtggaggcaccaagcttgaaatcaaa

SEQ ID NO: 9 - DNA, 360 bp, synthetic construct
  source 1..360: mol_type = other DNA
gaagtacagctggaggagtctgggggaggcttagtgaagcctggagggtccctgacactctcctgtgcagcctctggattcactttcagtaactatggcatgtcttgggttcgccagtctccagagaagaggctggagtgggtcgcagaaatcagtagtggtagtagctatatttactatccagacactgtgacgggccgattcaccatctccagagacaatgccaagaacaccctgtacctggaaatgaacagtctgaggtctgacgacacggccatgtattactgtgcaaggggtcctagtatgatttcgccgttctactttgactactggggccaaggcaccactctcacagtctcc

SEQ ID NO: 10 - DNA, 321 bp, synthetic construct
  source 1..321: mol_type = other DNA
gatattgtgctcacccagtctccagcatccctgtccgtggctacaggagaaaaagtcactatcagatgcataaccagcactgatattgatgatgatatgaactggtaccagcagaagccaggggaacctcctaagctccttatttcagaaggcaatactcttcgtcctggagtcccatcccgattctccagcagtggctatggcacagattttgtttttacaattgaaaacacgctctcagaagatgttgcagattactactgtttgcaaagtgataacatgccgtacacgttcggaggggggaccaagctggaaataaaa